(19) United States
(12) Patent Application Publication
(10) Pub. No.: US 2024/0307353 A1
(43) Pub. Date: Sep. 19, 2024

(54) JAK1 PATHWAY INHIBITORS FOR THE TREATMENT OF ASTHMA

(71) Applicant: Incyte Corporation, Wilmington, DE (US)

(72) Inventors: Kurt Andrew Brown, Narberth, PA (US); Irma Dzhindzhikhashvili, Givrins (CH); Cornelius P. Sailer, Leonia, NJ (US); Leandro Luiz Dos Santos, King of Prussia, PA (US)

(21) Appl. No.: 18/605,907

(22) Filed: Mar. 15, 2024

Related U.S. Application Data
(60) Provisional application No. 63/452,530, filed on Mar. 16, 2023.

(51) Int. Cl. A61K 31/4155; A61K 31/519; A61P 11/06

(52) U.S. Cl. CPC A61K 31/4155; A61K 31/519; A61P 11/06

(57) ABSTRACT

This disclosure relates to JAK1 pathway inhibitors and their use in treating asthma.

The present application claims the benefit of U.S. Provisional Application No. 63/452,530, filed Mar. 16, 2023, which is incorporated herein by reference in its entirety.

### TECHNICAL FIELD

This disclosure relates to JAK1 pathway inhibitors and the use thereof in treating asthma.

### BACKGROUND

Asthma is a chronic inflammatory airway disease that affects all age groups and is characterized by airway hyper-responsiveness. Asthma is a condition in which airways narrow and swell. Symptoms associated with airway obstruction include chest tightness, wheezing, cough, and dyspnea. Healthcare providers may identify asthma as intermittent asthma or persistent asthma. Intermittent asthma comes and goes so a subject can feel normal in between asthmatic episodes. Persistent asthma refers to subjects with symptoms most of the time. Asthma is one of the leading causes of morbidity worldwide, with an estimated global prevalence of asthma of 262 million cases in 2019 with 1-18% of the world population affected. The estimated prevalence of asthma is highest among Black people (11.2%), followed by White (7.6%), Hispanic (6.8%), and non-Hispanic (6.3%) people. Significant mortality is associated with asthma, resulting in 455,000 deaths worldwide and approximately 4,000 deaths in the United States in 2019.

Many cases of asthma remain uncontrolled despite the use of medium-to-high dose inhaled corticosteroids (ICS) in combination with a second controller medication (e.g., long-acting bronchodilator (LABA)). Some cases of asthma require systemic oral corticosteroids (OCS) as controller therapy for sustained symptom control. A systematic literature review found that among patients with moderate to severe asthma, the reported prevalence of uncontrolled asthma despite ICS-LABA treatment varied widely across studies (12.9%-100%), and when limited to larger cohort studies, the prevalence of uncontrolled asthma ranged from 15.4% to 75.1%. Long-term use of OCS therapies, independent of the dose, have been reported to elevate the risk of comorbidity and complications and is associated with a significant increase in healthcare resource utilization. Thus, there is an unmet need for new therapeutics, especially for patients with asthma that remains uncontrolled by ICS-LABA treatment.

### DESCRIPTION OF THE DRAWINGS

FIG. 1 depicts an outline of a phase 2 randomized, double-blind, placebo-controlled dose-ranging study of the efficacy and safety of Compound 1.

FIGS. 2A-C depict the effect of Compound 1 on IL-4, IL-5, and IL-13 cytokine levels in a whole blood assay.

FIGS. 3A-C depict the effect of Compound 1 on Th1 and Th17/Th22 cytokine levels in a whole blood assay.

FIGS. 4A-B depict the effect of Compound 1 on CXCL10 chemokine and IL-6 cytokine levels in a whole blood assay.

FIGS. 5A-B depict the effect of Compound 1 on eosinophil and neutrophil activation marker CD69.

FIGS. 6A-B depict the effect of Compound 1 on the elasticity of the lung (H).

FIGS. 7A-B depict the effect of Compound 1 on the airway resistance in small airways and alveoli (G).

### SUMMARY

Provided herein are methods for the treatment of asthma in a subject in need thereof, comprising administering to the subject a therapeutically effective amount of a JAK1 pathway inhibitor, or a pharmaceutically acceptable salt thereof.

Provided herein is a JAK1 pathway inhibitor, or a pharmaceutically acceptable salt thereof, for the treatment of asthma in a subject in need thereof.

Provided herein is a use of a JAK1 pathway inhibitor, or a pharmaceutically acceptable salt thereof, for manufacture of a medicament for use in treating asthma in a subject in need thereof. In some embodiments, the asthma is noneosinophilic asthma and/or eosinophilic asthma.

In some embodiments, a method for treating asthma in a subject includes administering to the subject a therapeutically effective amount of a JAK1 pathway inhibitor, or a pharmaceutically acceptable salt thereof, wherein the asthma is noneosinophilic asthma.

In some embodiments, the JAK1 pathway inhibitor, or a pharmaceutically acceptable salt thereof, is selective for JAK1 over JAK2, JAK3, and Tyk2.

In some embodiments, the JAK1 pathway inhibitor, or a pharmaceutically acceptable salt thereof, is selective for JAK1 over JAK2.

In some embodiments, the JAK1 pathway inhibitor is 4-[3-(cyanomethyl)-3-(3′,5′-dimethyl-1H,1′H-4,4′-bipyrazol-1-yl)azetidin-1-yl]-2,5-difluoro-N-[(1S)-2,2,2-trifluoro-1-methylethyl]benzamide, or a pharmaceutically acceptable salt thereof.

In some embodiments, the JAK1 pathway inhibitor is 4-[3-(cyanomethyl)-3-(3′,5′-dimethyl-1H,1′H-4,4′-bipyrazol-1-yl)azetidin-1-yl]-2,5-difluoro-N-[(1S)-2,2,2-trifluoro-1-methylethyl]benzamide phosphoric acid salt.

In some embodiments, the JAK1 pathway inhibitor, or pharmaceutically acceptable salt thereof, is administered in a daily dose of about 10 mg to about 80 mg on a free base basis.

In some embodiments, the JAK1 pathway inhibitor, or pharmaceutically acceptable salt thereof, is administered orally.

In some embodiments, the JAK1 pathway inhibitor, or pharmaceutically acceptable salt thereof, is administered orally via tablet.

In some embodiments, the JAK1 pathway inhibitor, or pharmaceutically acceptable salt thereof, is administered in a daily dose of about 15 mg, about 45 mg, or about 75 mg on a free base basis.

In some embodiments, the JAK1 pathway inhibitor, or pharmaceutically acceptable salt thereof, is administered in combination with a further therapeutic agent.

In some embodiments, the further therapeutic agent comprises a Janus kinase inhibitor.

In some embodiments, the Janus kinase inhibitor comprises ruxolitinib, or a pharmaceutically acceptable salt thereof.

In some embodiments, the administering comprises administering the JAK1 pathway inhibitor, or pharmaceutically acceptable salt thereof, together with at least one pharmaceutically acceptable carrier or excipient.

In some embodiments, the subject has about a 10% increase or greater in lung function after administration based on FEV1.

In some embodiments, the subject has a decrease in a number of inpatient hospitalization events due to asthma, wherein the decrease is about a 30%, about a 60%, or about a 90% reduction in the number of inpatient hospitalization events due to asthma.

In some embodiments, an the inpatient hospitalization event comprises admission to an inpatient facility or healthcare facility for ≥24 hours.

In some embodiments, the subject has about a 10% increase or greater in lung function after administration based on FVC.

In some embodiments, the subject has about a 10% improvement or greater in lung function after administration based on FeNO.

In some embodiments, the subject has about a 10% increase or greater in lung function after administration based on a PEF test result.

### DETAILED DESCRIPTION

The present invention provides, inter alia, a method of treating asthma in a subject in need thereof, comprising administering to the subject a therapeutically effective amount of a JAK1 pathway inhibitor, or a pharmaceutically acceptable salt thereof. In some embodiments, the present invention provides a method for treating asthma in a subject, said method comprising administering to the subject a therapeutically effective amount of a JAK1 pathway inhibitor, or a pharmaceutically acceptable salt thereof.

In some embodiments, a method for treating asthma in a subject comprises administering to the subject a therapeutically effective amount of a JAK1 pathway inhibitor, or a pharmaceutically acceptable salt thereof. In some embodiments, the asthma is noneosinophilic asthma and/or eosinophilic asthma.

In some embodiments, a method for treating asthma in a subject comprises administering to the subject a therapeutically effective amount of a JAK1 pathway inhibitor, or a pharmaceutically acceptable salt thereof, wherein the asthma is noneosinophilic asthma.

In some embodiments, a method for treating asthma in a subject comprises administering to the subject a therapeutically effective amount of a JAK1 pathway inhibitor, or a pharmaceutically acceptable salt thereof, wherein the asthma is eosinophilic asthma.

In some embodiments, a method for treating asthma in a subject comprises administering to the subject a therapeutically effective amount of a JAK1 pathway inhibitor, or a pharmaceutically acceptable salt thereof, wherein the asthma is eosinophilic asthma and noneosinophilic asthma.

In some embodiments, the JAK1 pathway inhibitor, or a pharmaceutically acceptable salt thereof, is selective for JAK1 over JAK2, JAK3, and Tyk2.

In some embodiments, the JAK1 pathway inhibitor, or a pharmaceutically acceptable salt thereof, inhibits JAK1 (50% inhibitory concentration≈9 nM), with approximately 52-fold greater selectivity for JAK1 versus JAK2 and a range of approximately 45-to >1000-fold selectivity over all JAK family members (JAK2, JAK3, and TYK2).

In some embodiments, the JAK1 pathway inhibitor is 4-[3-(cyanomethyl)-3-(3′,5′-dimethyl-1H,1′H-4,4′-bipyrazol-1-yl)azetidin-1-yl]-2,5-difluoro-N-[(1S)-2,2,2-trifluoro-1-methylethyl]benzamide (Compound 1), or a pharmaceutically acceptable salt thereof.

In some embodiments, the JAK1 pathway inhibitor is 4-[3-(cyanomethyl)-3-(3′,5′-dimethyl-1H,1′H-4,4′-bipyrazol-1-yl)azetidin-1-yl]-2,5-difluoro-N-[(1S)-2,2,2-trifluoro-1-methylethyl]benzamide phosphoric acid salt.

In some embodiments, the asthma is eosinophilic asthma.

In some embodiments, the asthma is noneosinophilic asthma.

In some embodiments, the JAK1 pathway inhibitor, or pharmaceutically acceptable salt thereof, is administered in a daily dose of about 5 mg to about 95 mg on a free base basis.

In some embodiments, the JAK1 pathway inhibitor, or pharmaceutically acceptable salt thereof, is administered in a daily dose of about 10 mg to about 80 mg on a free base basis.

In some embodiments, the JAK1 pathway inhibitor, or pharmaceutically acceptable salt thereof, is administered in a daily dose of about 15 mg, about 45 mg, or about 75 mg on a free base basis.

In some embodiments, the JAK1 pathway inhibitor, or pharmaceutically acceptable salt thereof, is administered in a daily dose of about 45 mg or about 75 mg on a free base basis.

In some embodiments, the JAK1 pathway inhibitor, or pharmaceutically acceptable salt thereof, is administered in a daily dose of about 45 mg on a free base basis.

In some embodiments, the JAK1 pathway inhibitor, or pharmaceutically acceptable salt thereof, is administered in a daily dose of about 75 mg on a free base basis.

In some embodiments, the JAK1 pathway inhibitor, or pharmaceutically acceptable salt thereof, is administered in a daily dose of about 30 mg on a free base basis.

In some embodiments, the JAK1 pathway inhibitor, or pharmaceutically acceptable salt thereof, is administered in a daily dose of about 60 mg on a free base basis.

In some embodiments, the JAK1 pathway inhibitor, or pharmaceutically acceptable salt thereof, is administered in combination with a further therapeutic agent.

In some embodiments, the further therapeutic agent comprises a Janus kinase inhibitor

In some embodiments, the Janus kinase inhibitor comprises ruxolitinib, or a pharmaceutically acceptable salt thereof.

In some embodiments, the administering comprises administering the JAK1 pathway inhibitor, or pharmaceutically acceptable salt thereof, together with at least one pharmaceutically acceptable carrier or excipient.

Asthma is heterogeneous in terms of both its response to treatment and the underlying pathophysiological pathways (endotypes) involved. 2 endotypes are broadly recognized:

eosinophilic (Type 2-high or Type 2) and noneosinophilic (Type 2-low or non-Type 2) asthma. In some embodiments, the asthma treated with compounds and methods described is eosinophilic asthma. The eosinophilic endotype is characterized by peripheral blood eosinophilia and infiltration of eosinophils in the airway. Eosinophilic endotype is associated with activation of cytokines derived from Th2 cells (e.g., IL-4, IL-5, and IL-13) driving the inflammatory process.

In some embodiments, the asthma treated with compounds and methods described is noneosinophilic asthma. In the noneosinophilic endotype, eosinophilia is not typically observed, but these patients often exhibit airway neutrophilia and inflammation that may be mediated through Th1 (IFN-γ, TNF, IL-1, and IL-6) and/or Th17 (IL-17A, IL-17E, IL-17F, and IL-22) responses.

Many asthma therapies, including most currently available biologics (e.g., monoclonal antibodies to cytokines or cytokine receptors), target Type 2 inflammation, which allows them to be effective against eosinophilic asthma but not the noneosinophilic endotype. Noneosinophilic asthma tends to respond poorly to CS therapy, possibly due to cytokine production by Th17 cells being resistant to inhibition by CS. Importantly, for both endotypes, cytokines and growth factors that signal via receptors coupled to JAK have been implicated in the mechanism of disease. Thus there is the possibility that targeting JAKs may provide a novel approach for treating both endotypes of asthma.

In some embodiments, efficacy of the treatment method disclosed herein can be established based upon percent change from various baseline measurements using various indicators. For example, lung function may be measured to track efficacy Lung function may be measured based on forced expiratory volume in the first 1 second (FEV₁). FEV₁, expressed as percent of the predicted normal (PNV), may be calculated as follows:

FEV₁ % of PNV=(FEV₁ measured/FEV₁PNV)×100.

Lung function may be measured based on forced vital capacity (FVC). Lung function may be measured using spirometry. The Global Lung Function Initiative equations will be used to determine the PNVs. In some embodiments, a JAK1 inhibitor, e.g., Compound 1, and/or methods of use described herein result in about a 5%, about a 10%, about a 20%, about a 30%, about a 40%, about a 50%, about a 60%, about a 70%, about a 80%, about a 90%, or about a 95% increase in lung function in a subject based on FEV₁ and/or FVC.

In some embodiments, efficacy of compounds and/or methods described herein are determined by monitoring a subject using fractional exhaled nitric oxide (FeNO). In some embodiments, a JAK1 inhibitor, e.g., Compound 1, and/or methods of use described herein result in about a 5%, about a 10%, about a 20%, about a 30%, about a 40%, about a 50%, about a 60%, about a 70%, about a 80%, about a 90%, or about a 95% improvement in a standardized single-breath FeNO test result in a subject.

Typically the FeNO test should be completed before spirometry since spirometry can potentially impact the nitric oxide measurement. Typically postbaseline FeNO tests should be performed within ±1.5 hours of the time that FeNO was performed on D1/BL. If a participant has had a respiratory infection in the 2 weeks prior to the FeNO test, then the FeNO should not be performed until >2 weeks after the infection. Participants should be instructed not to eat or drink 1 hour prior to the FeNO test.

In some embodiments, efficacy of compounds and/or methods described herein are determined by monitoring a subject using peak expiratory flow assessment (PEF). This testing may be done at home by a subject. In some embodiments, a JAK1 inhibitor, e.g., Compound 1, and/or methods of use described herein result in about a 5%, about a 10%, about a 20%, about a 30%, about a 40%, about a 50%, about a 60%, about a 70%, about a 80%, about a 90%, or about a 95% improvement in a PEF test result in a subject.

In some embodiments, efficacy of compounds and/or methods described herein are determined by monitoring a number of inpatient hospitalization events. In some embodiments, a JAK1 inhibitor, e.g., Compound 1, and/or methods of use described herein result in about a 5%, about a 10%, about a 20%, about a 30%, about a 40%, about a 50%, about a 60%, about a 70%, about a 80%, about a 90%, or about a 95% reduction in inpatient hospitalization events in a subject. In some embodiments, efficacy of compounds and/or methods described herein are determined by monitoring a number of inpatient hospitalization events. In some embodiments, a JAK1 inhibitor, e.g., Compound 1, and/or methods of use described herein result in about a 30%, about a 60%, or about a 90% reduction in inpatient hospitalization events in a subject. In some embodiments, a JAK1 inhibitor, e.g., Compound 1, and/or methods of use described herein may result in a reduction of an inpatient hospitalization event (defined as admission to an inpatient facility and/or evaluation and treatment in a healthcare facility for ≥24 hours) due to asthma.

Typically an electronic, handheld spirometer (peak flow meter) will be dispensed to the subject during the run-in period. Typically the subject will perform PEF testing in the morning upon awakening (and prior to taking their morning asthma controller) and in the evening (and prior to taking their evening asthma controller if taken BID). Subjects should perform 3 consecutive peak flow maneuvers while sitting or standing but in the same position at every testing. The highest of the 3 values will be recorded in the eDiary. When possible, PEF testing should be performed at least 6 hours after the last dose of short-acting beta-agonist or SABA rescue medication.

In some embodiments, efficacy of compounds and/or methods described herein are determined by monitoring patient-reported outcomes (PROs). This testing may be done at home or in clinic by a subject. In some embodiments, a JAK1 inhibitor, e.g., Compound 1, and/or methods of use described herein result in about a 5%, about a 10%, about a 20%, about a 30%, about a 40%, about a 50%, about a 60%, about a 70%, about a 80%, about a 90%, or about a 95% improvement in a PRO test result in a subject.

A PRO may include an asthma symptom diary (ASD). The ASD is a standardized, validated instrument consisting of 10 items: 5 items for the morning and 5 items for the evening. The morning items assess nighttime symptom severity related to wheezing, shortness of breath, cough, and chest tightness and the frequency of nighttime awakening. The evening items assess symptom severity related to wheezing, shortness of breath, cough, chest tightness, and activity limitation since waking. Each item may be scored using a 5-point categorical response scale ranging from 0 (no symptom, no nighttime awakening, or no activity limitation) to 4 (very severe symptom, unable to sleep, or extreme activity limitation). The daily ASD score is the mean response to all 10 questions and is calculated using data from the evening diary assessment and the subsequent morning diary assessment. If at least 4 of the 7 daily ASD scores are available, the 7-day average asthma symptom score will be calculated with no imputation.

A PRO may include an asthma control questionnaire-6 items (ACQ-6). The ACQ-6 may include a standardized, validated questionnaire that will be used to collect data on the adequacy of asthma control or change in asthma control following treatment. The ACQ-6 may consist of 6 items that evaluate asthma symptoms (5 items) and rescue bronchodilator (BD) use (1 item) using a recall period of the previous 1 week. The asthma symptoms evaluated are nighttime waking, symptoms on waking, activity limitation, shortness of breath, and wheezing. For each of the 6 items, the questionnaire uses a 7-point categorical response scale ranging from 0 (e.g., no impairment) to 6 (e.g., maximum impairment); the wording for the response associated with each number varies based on the question asked. The mean ACQ-6 score is the mean response to all 6 items. In some embodiments, by omitting the ACQ-6 BD use question from the scoring algorithm, an ACQ-5 score will also be calculated.

A PRO may include a standardized asthma quality of life questionnaire (AQLQ(S)). The AQLQ(S) may include a standardized, validated instrument that will be used to collect data on functional problems that are troublesome for most adults with asthma. The AQLQ(S) consists of 32 items grouped into 4 domains (symptoms, activity limitation, emotional function, and environmental stimuli), and it uses a recall period of the previous 2 weeks. For each of the 32 items, there is an 8-point categorical response scale ranging from 7 (not impaired) to 1 (severely impaired). The overall score is the mean response to all 32 questions, and the individual domain scores are the mean response to all items within that domain.

A PRO may include a EuroQol 5-dimension 5-level scale (EQ-SD-5L). The EQ-5D-5L may include a standardized instrument for use as a measure of health outcomes. The EQ-5D-5L will provide data for use in economic models and analyses, including developing health utilities or quality-adjusted life years. The EQ-5D-5L consists of 2 sections: the EQ-5D descriptive system and the EQ VAS, which asks about the participant's health for that day. The descriptive system comprises 5 dimensions: mobility, self-care, usual activities, pain/discomfort, and anxiety/depression. Each dimension has 5 levels: no problems, slight problems, moderate problems, severe problems, and extreme problems. The EQ VAS records the participant's self-rated health on a vertical VAS (0-100), where the anchors are labeled as “The best health you can imagine” and “The worst health you can imagine.”

A PRO may include a work productivity and activity impairment questionnaire: asthma (WPAI:Asthma). The WPAI: Asthma is a standardized, validated instrument that will provide data on participants' work impairment due to asthma based on the participant's self-reported productivity loss. The WPAI: Asthma consists of 6 questions, with the first question used to determine whether the completer is currently employed, and the remaining 5 questions assessing the impact of asthma on work over the past 7 days.

A PRO may include a patient global impression of severity (PGI-S). The PGI-S will provide data on asthma symptom severity from the participant's perspective. The PGI-S is a single-item questionnaire to evaluate disease severity. Participants will rate their asthma symptoms experienced at each study visit using a 5-point scale (none, mild, moderate, severe, very severe).

A PRO may include a clinician and patient global impression of change (CGI-C and PGI-C). The CGI-C and PGI-C instruments will provide data on the overall response to treatment from the investigator's and participant's perspective, respectively. The CGI-C (investigator-completed) and PGI-C (subject-completed) are single-item questionnaires about the degree of change in the participant's overall asthma status compared with the start of treatment. Both use a 7-point categorical response scale ranging from 1 (very much improved) to 7 (very much worse)

A PRO may include a sino-nasal outcome test (SNOT-22). Chronic rhinosinusitis is a common comorbidity for patients with severe asthma that may benefit from JAK inhibition. Therefore, SNOT-22, a standardized, validated instrument, will be completed to collect data on the impact of chronic rhinosinusitis on the participants' quality of life as well as to measure outcomes following treatment with compounds disclosed herein. The SNOT-22 consists of 22 items (each listed as a specific symptom), and it uses a recall period of the previous 2 weeks. For each of the 22 items, there is a 6-point categorical response scale ranging from 0 (no problem) to 5 (problem as bad as it can be) plus an additional column where the participant can mark up to 5 symptoms considered as the “most important items.” The total score is a sum of all items (ranging from 0 to 110), with higher scores indicating poorer outcomes.

The methods described herein utilize JAK1 pathway inhibitors, particularly JAK1 selective inhibitors. A JAK1 selective inhibitor is a compound that inhibits JAK1 activity preferentially over other Janus kinases JAK1 plays a central role in a number of cytokine and growth factor signaling pathways that, when dysregulated, can result in or contribute to disease states. In other autoimmune diseases and cancers, elevated systemic levels of inflammatory cytokines that activate JAK1 may also contribute to the disease and/or associated symptoms. Therefore, patients with autoimmune diseases like asthma may benefit from JAK1 inhibition. Selective inhibitors of JAK1 may be efficacious while avoiding unnecessary and potentially undesirable effects of inhibiting other JAK kinases.

In some embodiments, the JAK1 pathway inhibitor, or a pharmaceutically acceptable salt thereof, is selective for JAK1 over JAK2, JAK3, and TYK2 (i.e., a JAK1 selective inhibitor). For example, the compounds described herein, or pharmaceutically acceptable salts thereof, preferentially inhibit JAK1 over one or more of JAK2, JAK3, and TYK2. In some embodiments, the compounds inhibit JAK1 preferentially over JAK2 (e.g., have a JAK2/JAK1 IC₅₀ ratio >1). In some embodiments, the compounds or salts are about 10-fold more selective for JAK1 over JAK2. In some embodiments, the compounds or salts are about 3-fold, about 5-fold, about 10-fold, about 15-fold, or about 20-fold more selective for JAK1 over JAK2 as calculated by measuring IC₅₀ at 1 mM ATP (e.g., see Example A).

In some embodiments, the JAK1 pathway inhibitor is a compound of Table 1, or a pharmaceutically acceptable salt thereof. The compounds in Table 1 are selective JAK1 inhibitors (selective over JAK2, JAK3, and TYK2). The IC50 values obtained by the method of Example A at 1 mM ATP are shown in Table 1.

The compounds of Table 1 can be prepared by the synthetic procedures described, for example, in US Patent Publ. No. 2011/0224190, filed Mar. 9, 2011, US Patent Publ. No. 2014/0343030, filed May 16, 2014, US Patent Publ. No. 2014/0121198, filed Oct. 31, 2013, US Patent Publ. No. 2010/0298334, filed May 21, 2010, US Patent Publ. No. 2011/0059951, filed Aug. 31, 2010, US Patent Publ. No. 2012/0149681, filed Nov. 18, 2011, US Patent Publ. No. 2012/0149682, filed Nov. 18, 2011, US Patent Publ. 2013/0018034, filed Jun. 19, 2012, US Patent Publ. No. 2013/0045963, filed Aug. 17, 2012, and US Patent Publ. No. 2014/0005166, filed May 17, 2013, each of which is incorporated herein by reference in its entirety.

TABLE 1

| | | | | JAK1 | |
|---|---|---|---|---|---|
| Comp. | | | | IC₅₀ | JAK2/ |
| No. | Prep. | Name | Structure | (nM) | JAK1 |
| 1 | US 2014/0343030 (Example 7) | 4-[3-(cyanomethyl)-3-(3′,5′- dimethyl-1H,1′H-4,4′- bipyrazol-1-yl)azetidin-1- yl]-2,5-difluoro-N-[(1S)- 2,2,2-trifluoro-1- methylethyl]benzamide | 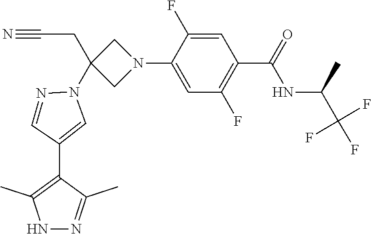 [SMILES: Cc1n[nH]c(C)c1-c1cnn(C2(CC#N)CN(c3cc(F)c(C(=O)N[C@@H](C)C(F)(F)F)cc3F)C2)c1] | +++ | >10 |
| 2 | US 2011/ 0224190 (Example 154) | 4-{3-(Cyanomethyl)-3-[4- (7H-pyrrolo[2,3- d]pyrimidin-4-yl)-1H- pyrazol-1-yl]azetidin-1-yl}- N-[4-fluoro-2- (trifluoromethyl)phenyl] piperidine-1-carboxamide | 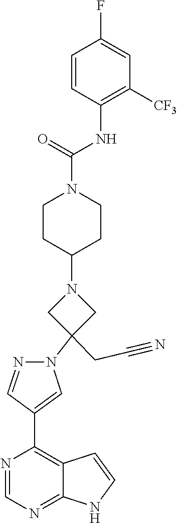 [SMILES: N#CCC1(n2cc(-c3ncnc4[nH]ccc34)cn2)CN(C2CCN(C(=O)Nc3ccc(F)cc3C(F)(F)F)CC2)C1] | + | >10 |
| 3 | US 2011/ 0224190 (Example 85) | [3-[4-(7H-pyrrolo[2,3- d]pyrimidin-4-yl)-1H- pyrazol-1-yl]-1-(1-{[2- (trifluoromethyl)pyrimidin- 4-yl]carbonyl}piperidin-4- yl)azetidin-3-yl]acetonitrile | 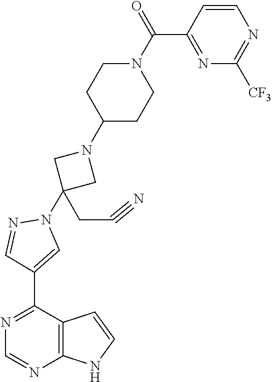 [SMILES: N#CCC1(n2cc(-c3ncnc4[nH]ccc34)cn2)CN(C2CCN(C(=O)c3ccnc(C(F)(F)F)n3)CC2)C1] | + | >10 |
| 4 | US 2011/ 0224190 (Example 1) | {1-{1-[3-Fluoro-2- (trifluoromethyl)isonicotinoyl] piperidin-4-yl}-3-[4- (7H-pyrrolo[2,3- d]pyrimidin-4-yl)-1H- pyrazol-1-yl]azetidin-3- yl}acetonitrile | 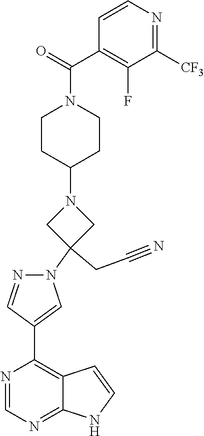 [SMILES: N#CCC1(n2cc(-c3ncnc4[nH]ccc34)cn2)CN(C2CCN(C(=O)c3ccnc(C(F)(F)F)c3F)CC2)C1] | + | >10 |
| 5 | US 2014/0121198 (Example 20) | ((2R,5S)-5-{2-[(1R)-1- hydroxyethyl]-1H- imidazo[4,5-d]thieno[3,2- b]pyridin-1-yl}tetrahydro- 2H-pyran-2-yl)acetonitrile | 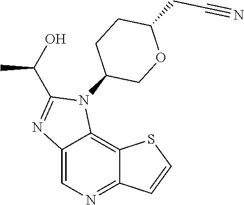 [SMILES: C[C@@H](O)c1nc2cnc3ccsc3c2n1[C@H]1CC[C@H](CC#N)OC1] | ++ | >10 |
| 6 | US 2010/ 0298334 (Example 2)^{a} | 3-[1-(6-chloropyridin-2- yl)pyrrolidin-3-yl]-3-[4- (7H-pyrrolo[2,3- d]pyrimidin-4-yl)-1H- pyrazol-1-yl]propanenitrile | 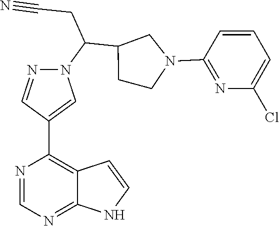 [SMILES: N#CCC(C1CCN(c2cccc(Cl)n2)C1)n1cc(-c2ncnc3[nH]ccc23)cn1] | + | >10 |
| 7 | US 2010/ 0298334 (Example 13c) | 3-(1-[1,3]oxazolo[5,4- b]pyridin-2-ylpyrrolidin-3- yl)-3-[4-(7H-pyrrolo[2,3- d]pyrimidin-4-yl)-1H- pyrazol-1-yl]propanenitrile | 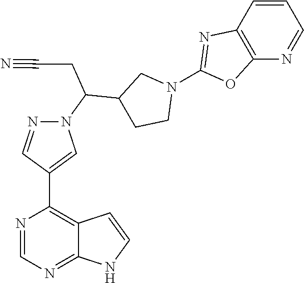 [SMILES: N#CCC(C1CCN(c2nc3cccnc3o2)C1)n1cc(-c2ncnc3[nH]ccc23)cn1] | + | >10 |
| 8 | US 2011/ 0059951 (Example 12) | 4-[(4-{3-cyano-2-[4-(7H- pyrrolo[2,3-d]pyrimidin-4- yl)-1H-pyrazol-1- yl]propyl}piperazin-1- yl)carbonyl]-3- fluorobenzonitrile | 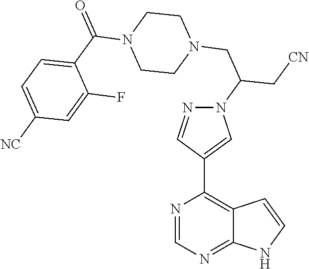 [SMILES: N#CCC(CN1CCN(C(=O)c2ccc(C#N)cc2F)CC1)n1cc(-c2ncnc3[nH]ccc23)cn1] | + | >10 |
| 9 | US 2011/ 0059951 (Example 13) | 4-[(4-{3-cyano-2-[3-(7H- pyrrolo[2,3-d]pyrimidin-4- yl)-1H-pyrrol-1- yl]propyl}piperazin-1- yl)carbonyl]-3- fluorobenzonitrile | 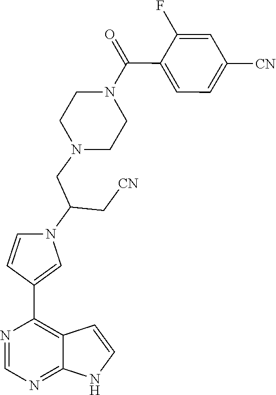 [SMILES: N#CCC(CN1CCN(C(=O)c2ccc(C#N)cc2F)CC1)n1ccc(-c2ncnc3[nH]ccc23)c1] | + | >10 |
| 10 | US 2012/ 0149681 (Example 7b) | [trans-1-[4-(7H- pyrrolo[2,3-d]pyrimidin-4- yl)-1H-pyrazol-1-yl]-3-(4- {[2- (trifluoromethyl)pyrimidin- 4-yl]carbonyl}piperazin-1- yl)cyclobutyl]acetonitrile | 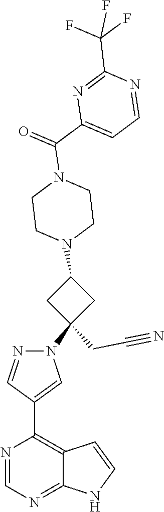 [SMILES: N#CC[C@]1(n2cc(-c3ncnc4[nH]ccc34)cn2)C[C@H](N2CCN(C(=O)c3ccnc(C(F)(F)F)n3)CC2)C1] | + | >10 |
| 11 | US 2012/ 0149681 (Example 157) | {trans-3-(4-{[4-[(3- hydroxyazetidin-1- yl)methyl]-6- (trifluoromethyl)pyridin-2- yl]oxy}piperidin-1-yl)-1- [4-(7H-pyrrolo[2,3- d]pyrimidin-4-yl)-1H- pyrazol-1- yl]cyclobutyl}acetonitrile | 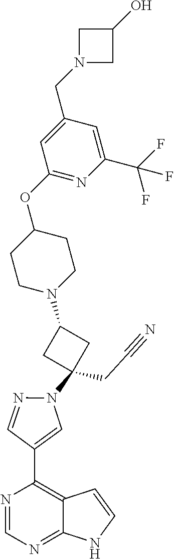 [SMILES: N#CC[C@]1(n2cc(-c3ncnc4[nH]ccc34)cn2)C[C@H](N2CCC(Oc3cc(CN4CC(O)C4)cc(C(F)(F)F)n3)CC2)C1] | + | >10 |
| 12 | US 2012/ 0149681 (Example 161) | {trans-3-(4-{[4-{[(2S)-2- (hydroxymethyl)pyrrolidin- 1-yl]methyl}-6- (trifluoromethyl)pyridin-2- yl]oxy}piperidin-1-yl)-1- [4-(7H-pyrrolo[2,3- d]pyrimidin-4-yl)-1H- pyrazol-1- yl]cyclobutyl}acetonitrile | 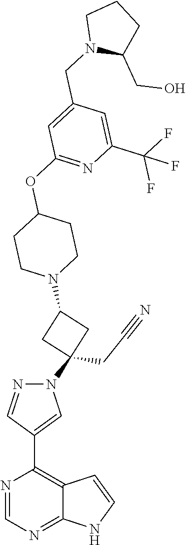 [SMILES: N#CC[C@]1(n2cc(-c3ncnc4[nH]ccc34)cn2)C[C@H](N2CCC(Oc3cc(CN4CCC[C@H]4CO)cc(C(F)(F)F)n3)CC2)C1] | + | >10 |
| 13 | US 2012/ 0149681 (Example 162) | {trans-3-(4-{[4-{[(2R)-2- (hydroxymethyl)pyrrolidin- 1-yl]methyl}-6- (trifluoromethyl)pyridin-2- yl]oxy}piperidin-1-yl)-1- [4-(7H-pyrrolo[2,3- d]pyrimidin-4-yl)-1H- pyrazol-1- yl]cyclobutyl}acetonitrile | 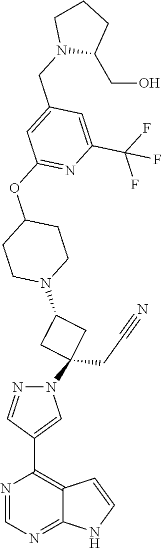 [SMILES: N#CC[C@]1(n2cc(-c3ncnc4[nH]ccc34)cn2)C[C@H](N2CCC(Oc3cc(CN4CCC[C@@H]4CO)cc(C(F)(F)F)n3)CC2)C1] | + | >10 |
| 14 | US 2012/ 0149682 (Example 20)^{b} | 4-(4-{3- [(dimethylamino)methyl]- 5-fluorophenoxy}piperidin- 1-yl)-3-[4-(7H-pyrrolo[2,3- d]pyrimidin-4-yl)-1H- pyrazol-1-yl]butanenitrile | 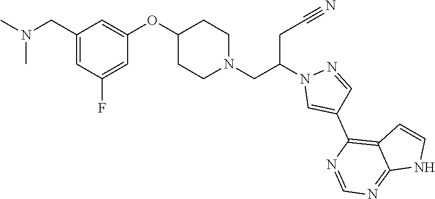 [SMILES: CN(C)Cc1cc(F)cc(OC2CCN(CC(CC#N)n3cc(-c4ncnc5[nH]ccc45)cn3)CC2)c1] | + | >10 |
| 15 | US 2013/ 0018034 (Example 18) | 5-{3-(cyanomethyl)-3-[4- (7H-pyrrolo[2,3- d]pyrimidin-4-yl)-1H- pyrazol-1-yl]azetidin-1-yl}- N-isopropylpyrazine-2- carboxamide | 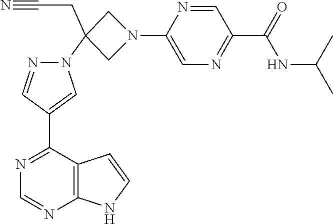 [SMILES: CC(C)NC(=O)c1cnc(N2CC(CC#N)(n3cc(-c4ncnc5[nH]ccc45)cn3)C2)cn1] | + | >10 |
| 16 | US 2013/ 0018034 (Example 28) | 4-{3-(cyanomethyl)-3-[4- (7H-pyrrolo[2,3- d]pyrimidin-4-yl)-1H- pyrazol-1-yl]azetidin-1-yl}- 2,5-difluoro-N-[(1S)-2,2,2- trifluoro-1- methylethyl]benzamide | 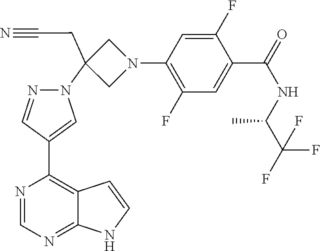 [SMILES: C[C@H](NC(=O)c1cc(F)c(N2CC(CC#N)(n3cc(-c4ncnc5[nH]ccc45)cn3)C2)cc1F)C(F)(F)F] | + | >10 |
| 17 | US 2013/ 0018034 (Example 34) | 5-{3-(cyanomethyl)-3-[4- (1H-pyrrolo[2,3-b]pyridin- 4-yl)-1H-pyrazol-1- yl]azetidin-1-yl}-N- isopropylpyrazine-2- carboxamide | 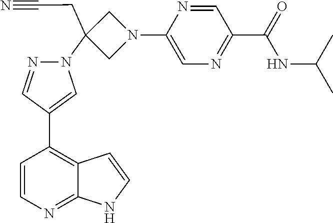 [SMILES: CC(C)NC(=O)c1cnc(N2CC(CC#N)(n3cc(-c4ccnc5[nH]ccc45)cn3)C2)cn1] | + | >10 |
| 18 | US 2013/ 0045963 (Example 45) | {1-(cis-4-{[6-(2- hydroxyethyl)-2- (trifluoromethyl)pyrimidin- 4-yl]oxy}cyclohexyl)-3-[4- (7H-pyrrolo[2,3- d]pyrimidin-4-yl)-1H- pyrazol-1-yl]azetidin-3- yl}acetonitrile | 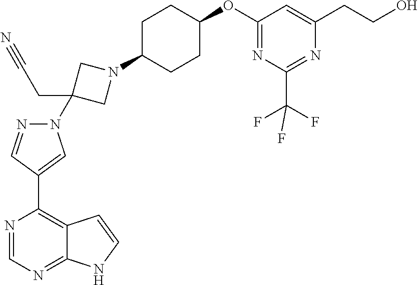 [SMILES: N#CCC1(n2cc(-c3ncnc4[nH]ccc34)cn2)CN([C@H]2CC[C@@H](Oc3cc(CCO)nc(C(F)(F)F)n3)CC2)C1] | + | >10 |
| 19 | US 2013/ 0045963 (Example 65) | {1-(cis-4-{[4- [(ethylamino)methyl]-6- (trifluoromethyl)pyridin-2- yl]oxy}cyclohexyl)-3-[4- (7H-pyrrolo[2,3- d]pyrimidin-4-yl)-1H- pyrazol-1-yl]azetidin-3- yl}acetonitrile | 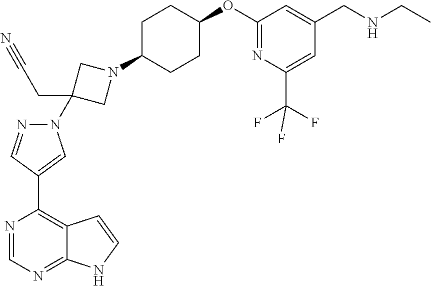 [SMILES: CCNCc1cc(O[C@H]2CC[C@@H](N3CC(CC#N)(n4cc(-c5ncnc6[nH]ccc56)cn4)C3)CC2)nc(C(F)(F)F)c1] | + | >10 |
| 20 | US 2013/ 0045963 (Example 69) | {1-(cis-4-{[4-(1-hydroxy-1- methylethyl)-6- (trifluoromethyl)pyridin-2- yl]oxy}cyclohexyl)-3-[4- (7H-pyrrolo[2,3- d]pyrimidin-4-yl)-1H- pyrazol-1-yl]azetidin-3- yl}acetonitrile | 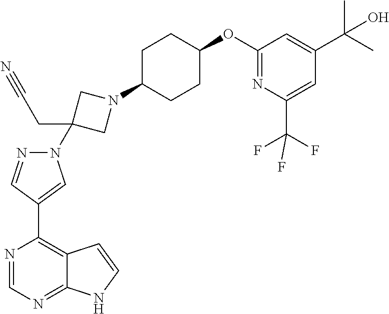 [SMILES: CC(C)(O)c1cc(O[C@H]2CC[C@@H](N3CC(CC#N)(n4cc(-c5ncnc6[nH]ccc56)cn4)C3)CC2)nc(C(F)(F)F)c1] | + | >10 |
| 21 | US 2013/ 0045963 (Example 95) | {1-(cis-4-{[4-{[(3R)-3- hydroxypyrrolidin-1- yl]methyl}-6- (trifluoromethyl)pyridin-2- yl]oxy}cyclohexyl)-3-[4- (7H-pyrrolo[2,3- d]pyrimidin-4-yl)-1H- pyrazol-1-yl]azetidin-3- yl}acetonitrile | 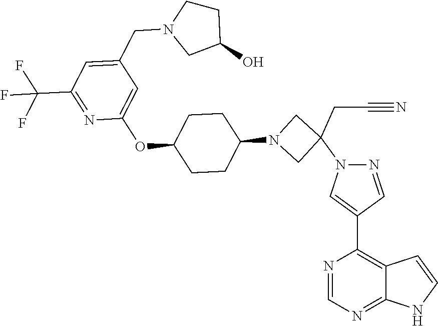 [SMILES: N#CCC1(n2cc(-c3ncnc4[nH]ccc34)cn2)CN([C@H]2CC[C@@H](Oc3cc(CN4CC[C@@H](O)C4)cc(C(F)(F)F)n3)CC2)C1] | + | >10 |
| 22 | US 2013/ 0045963 (Example 95) | {1-(cis-4-{[4-{[(3S)-3- hydroxypyrrolidin-1- yl]methyl}-6- (trifluoromethyl)pyridin-2- yl]oxy}cyclohexyl)-3-[4- (7H-pyrrolo[2,3- d]pyrimidin-4-yl)-1H- pyrazol-1-yl]azetidin-3- yl}acetonitrile | 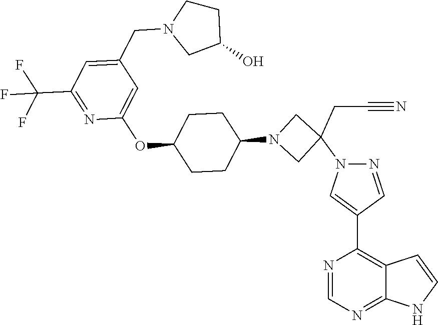 [SMILES: N#CCC1(n2cc(-c3ncnc4[nH]ccc34)cn2)CN([C@H]2CC[C@@H](Oc3cc(CN4CC[C@H](O)C4)cc(C(F)(F)F)n3)CC2)C1] | + | >10 |
| 23 | US 2014/ 0005166 (Example 1) | {trans-3-(4-{[4-({[(1S)-2- hydroxy-1- methylethyl]amino}methyl)- 6-(trifluoromethyl)pyridin- 2-yl]oxy}piperidin-1-yl)-1- [4-(7H-pyrrolo[2,3- d]pyrimidin-4-yl)-1H- pyrazol-1- yl]cyclobutyl}acetonitrile | 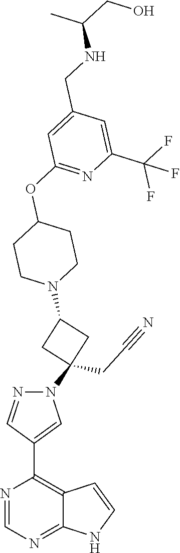 [SMILES: C[C@@H](CO)NCc1cc(OC2CCN([C@H]3C[C@](CC#N)(n4cc(-c5ncnc6[nH]ccc56)cn4)C3)CC2)nc(C(F)(F)F)c1] | + | >10 |
| 24 | US 2014/ 0005166 (Example 14) | {trans-3-(4-{[4-({[(2R)-2- hydroxypropyl]amino}methyl)- 6- (trifluoromethyl)pyridin-2- yl]oxy}piperidin-1-yl)-1- [4-(7H-pyrrolo[2,3- d]pyrimidin-4-yl)-1H- pyrazol-1- yl]cyclobutyl}acetonitrile | 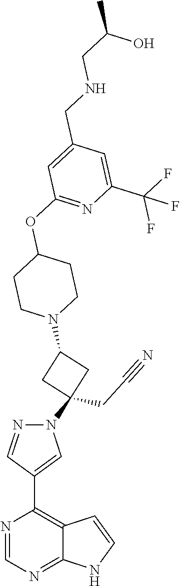 [SMILES: C[C@@H](O)CNCc1cc(OC2CCN([C@H]3C[C@](CC#N)(n4cc(-c5ncnc6[nH]ccc56)cn4)C3)CC2)nc(C(F)(F)F)c1] | + | >10 |
| 25 | US 2014/ 0005166 (Example 15) | {trans-3-(4-{[4-({[(2S)-2- hydroxypropyl]amino}methyl)- 6- (trifluoromethyl)pyridin-2- yl]oxy}piperidin-1-yl)-1- [4-(7H-pyrrolo[2,3- d]pyrimidin-4-yl)-1H- pyrazol-1- yl]cyclobutyl}acetonitrile | 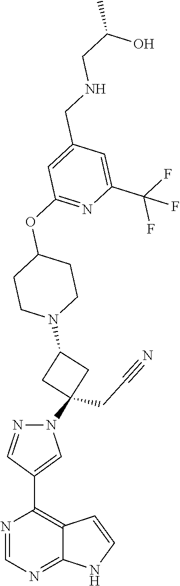 [SMILES: C[C@H](O)CNCc1cc(OC2CCN([C@H]3C[C@](CC#N)(n4cc(-c5ncnc6[nH]ccc56)cn4)C3)CC2)nc(C(F)(F)F)c1] | + | >10 |
| 26 | US 2014/ 0005166 (Example 20) | {trans-3-(4-{[4-(2- hydroxyethyl)-6- (trifluoromethyl)pyridin-2- yl]oxy}piperidin-1-yl)-1- [4-(7H-pyrrolo[2,3- d]pyrimidin-4-yl)-1H- pyrazol-1- yl]cyclobutyl}acetonitrile | 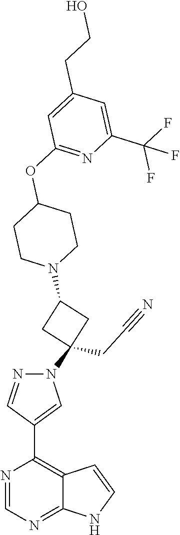 [SMILES: N#CC[C@]1(n2cc(-c3ncnc4[nH]ccc34)cn2)C[C@H](N2CCC(Oc3cc(CCO)cc(C(F)(F)F)n3)CC2)C1] | + | >10 |

+ means <10 nM (see Example A for assay conditions)
++ means ≤100 nM (see Example A for assay conditions)
+++ means ≤300 nM (see Example A for assay conditions)
^{a}Data for enantiomer 1
^{b}Data for enantiomer 2

In some embodiments, the JAK1 pathway inhibitor is 4-[3-(cyanomethyl)-3-(3′,5′-dimethyl-1H,1′H-4,4′-bipyrazol-1-yl)azetidin-1-yl]-2,5-difluoro-N-[(1S)-2,2,2-trifluoro-1-methylethyl]benzamide (Compound 1), or a pharmaceutically acceptable salt thereof. In some embodiments, the JAK1 pathway inhibitor is 4-[3-(cyanomethyl)-3-(3′,5′-dimethyl-1H,1′H-4,4′-bipyrazol-1-yl)azetidin-1-yl]-2,5-difluoro-N-[(1S)-2,2,2-trifluoro-1-methylethyl]benzamide phosphoric acid salt. Compound 1, and its salts, can be made by the procedures described in, e.g., U.S. Pat. No. 9,382,231 (see, e.g., Example 7), filed May 16, 2014, which is incorporated herein by reference in its entirety.

In some embodiments, the JAK1 pathway inhibitor is selected from the compounds, or pharmaceutically acceptable salts thereof, described in US Patent Publ. No. 2011/0224190, filed Mar. 9, 2011, US Patent Publ. No. 2014/0343030, filed May 16, 2014, US Patent Publ. No. 2014/0121198, filed Oct. 31, 2013, US Patent Publ. No. 2010/0298334, filed May 21, 2010, US Patent Publ. No. 2011/0059951, filed Aug. 31, 2010, US Patent Publ. No. 2012/0149681, filed Nov. 18, 2011, US Patent Publ. No. 2012/0149682, filed Nov. 18, 2011, US Patent Publ. 2013/0018034, filed Jun. 19, 2012, US Patent Publ. No. 2013/0045963, filed Aug. 17, 2012, and US Patent Publ. No. 2014/0005166, filed May 17, 2013, each of which is incorporated herein by reference in its entirety.

In some embodiments, the JAK1 pathway inhibitor is a compound of Formula I

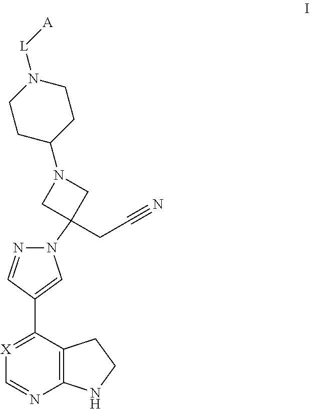

or a pharmaceutically acceptable salt thereof, wherein:
X is N or CH;
L is C(═O) or C(═O)NH;
A is phenyl, pyridinyl, or pyrimidinyl each of which is optionally substituted with 1 or 2 independently selected R¹ groups; and
each R¹ is, independently, fluoro, or trifluoromethyl.

In some embodiments, the compound of Formula I is {1-{1-[3-fluoro-2-(trifluoromethyl)isonicotinoyl]piperidin-4-yl}-3[4-(7H-pyrrolo[2,3-d]pyrimidin-4-yl)-1H-pyrazol-1-yl]azetidin-3-yl}acetonitrile, or a pharmaceutically acceptable salt thereof.

In some embodiments, the compound of Formula I is 4-{3-(Cyanomethyl)-3-[4-(7H-pyrrolo[2,3-d]pyrimidin-4-yl)-1H-pyrazol-1-yl]azetidin-1-yl}-N-[4-fluoro-2-(trifluoromethyl)phenyl]piperidine- 1-carboxamide, or a pharmaceutically acceptable salt thereof.

In some embodiments, the compound of Formula I is [3-[4-(7H-pyrrolo[2,3-d]pyrimidin-4-yl)-1H-pyrazol-1-yl]-1-(1-{[2-(trifluoromethyl)pyrimidin-4-yl]carbonyl}piperidin-4-yl)azetidin-3-yl]acetonitrile, or a pharmaceutically acceptable salt thereof.

In some embodiments, the JAK1 pathway inhibitor is a compound of Formula II

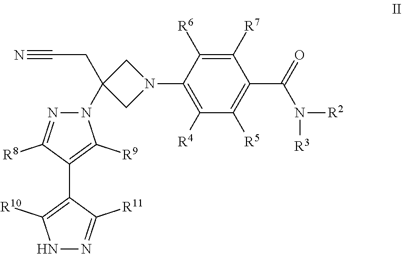

or a pharmaceutically acceptable salt thereof, wherein:
R² is C₁₋₆ alkyl, C₁₋₆ haloalkyl, C₃₋₆ cycloalkyl, or C₃₋₆ cycloalkyl-C₁₋₃ alkyl, wherein said C₁₋₆ alkyl, C₃₋₆ cycloalkyl, and C₃₋₆ cycloalkyl-C₁₋₃ alkyl, are each optionally substituted with 1, 2, or 3 substituents independently selected from fluoro, —CF₃, and methyl;
R³ is H or methyl;
R⁴ is H, F, or Cl;
R⁵ is H or F;
R⁶ is H or F;
R⁷ is H or F;
R⁸ is H or methyl;
R⁹ is H or methyl;
R¹⁰ is H or methyl; and
R¹¹ is H or methyl.

In some embodiments, the JAK1 pathway inhibitor is a compound of Formula III

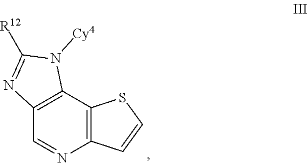

or a pharmaceutically acceptable salt thereof, wherein:
Cy⁴ is a tetrahydro-2H-pyran ring, which is optionally substituted with 1 or 2 groups independently selected from CN, OH, F, Cl, C₁₋₃ alkyl, C₁₋₃ haloalkyl, cyano-C₁₋₃ alkyl, HO-C₁₋₃ alkyl, amino, C₁₋₃ alkylamino, and di(C₁₋₃ alkyl)amino, wherein said C₁₋₃ alkyl and di(C₁₋₃ alkyl)amino is optionally substituted with 1, 2, or 3 substituents independently selected from F, Cl, C₁₋₃ alkylaminosulfonyl, and C₁₋₃ alkylsulfonyl; and
R¹² is —CH₂—OH, —CH(CH₃)—OH, or —CH₂—NHSO₂CH₃.

In some embodiments, the compound of Formula III is ((2R,5S)-5-{2-[(1R)-1-hydroxyethyl]-1H-imidazo[4,5-d]thieno[3,2-b]pyridin-1-yl}tetrahydro-2H-pyran-2-yl)acetonitrile, or a pharmaceutically acceptable salt thereof.

In some embodiments, the JAK1 pathway inhibitor, or a pharmaceutically acceptable salt thereof, is administered in a daily amount of from about 10 mg to about 80 mg on a free base basis. In some embodiments, the JAK1 pathway inhibitor, or a pharmaceutically acceptable salt thereof, is administered in a daily amount of about 75 mg on a free base basis. In some embodiments, the JAK1 pathway inhibitor, or a pharmaceutically acceptable salt thereof, is administered in a daily amount of about 45 mg on a free base basis. In some embodiments, the JAK1 pathway inhibitor, or a pharmaceutically acceptable salt thereof, is administered in a daily amount of about 15 mg on a free base basis.

The term “about” means “approximately” (e.g., plus or minus approximately 10% of the indicated value).

In some embodiments, the JAK1 pathway inhibitor, or a pharmaceutically acceptable salt thereof, is administered as one or more sustained release dosage forms each comprising the JAK1 pathway inhibitor, or a pharmaceutically acceptable salt thereof.

In some embodiments, the JAK1 pathway inhibitor, or a pharmaceutically acceptable salt thereof, is administered orally.

The embodiments described herein are intended to be combined in any suitable combination as if the embodiments are multiply dependent claims (e.g., the embodiments related to the selective JAK1 pathway inhibitor and doses of the same, the embodiments related to any salt forms of the compounds disclosed herein, the embodiments related to the individual types of cytokine related diseases or disorders, and the embodiments related to composition and/or administration can be combined in any combination).

All possible combinations are not separately listed herein merely for the sake of brevity.

The compounds described herein can be asymmetric (e.g., having one or more stercocenters). All stereoisomers, such as enantiomers and diastereomers, are intended unless otherwise indicated. Compounds that contain asymmetrically substituted carbon atoms can be isolated in optically active or racemic forms. Methods on how to prepare optically active forms from optically inactive starting materials are known in the art, such as by resolution of racemic mixtures or by stereoselective synthesis. Many geometric isomers of olefins, C═N double bonds, and the like can also be present in the compounds described herein, and all such stable isomers are contemplated in the present invention. Cis and trans geometric isomers of the compounds of the present invention are described and may be isolated as a mixture of isomers or as separated isomeric forms.

In some embodiments, the compound has the (R)-configuration. In some embodiments, the compound has the (S)-configuration.

Resolution of racemic mixtures of compounds can be carried out by any of numerous methods known in the art. An example method includes fractional recrystallizaion using a chiral resolving acid which is an optically active, salt-forming organic acid. Suitable resolving agents for fractional recrystallization methods are, for example, optically active acids, such as the D and L forms of tartaric acid, diacetyltartaric acid, dibenzoyltartaric acid, mandelic acid, malic acid, lactic acid or the various optically active camphorsulfonic acids such as β-camphorsulfonic acid. Other resolving agents suitable for fractional crystallization methods include stereoisomerically pure forms of α-methylbenzylamine (e.g., S and R forms, or diastereomerically pure forms), 2-phenylglycinol, norephedrine, ephedrine, N-methylephedrine, cyclohexylethylamine, 1,2-diaminocyclohexane, and the like.

Resolution of racemic mixtures can also be carried out by elution on a column packed with an optically active resolving agent (e.g., dinitrobenzoylphenylglycine). Suitable elution solvent composition can be determined by one skilled in the art.

Compounds described herein also include tautomeric forms. Tautomeric forms result from the swapping of a single bond with an adjacent double bond together with the concomitant migration of a proton. Tautomeric forms include prototropic tautomers which are isomeric protonation states having the same empirical formula and total charge. Example prototropic tautomers include ketone-enol pairs, amide-imidic acid pairs, lactam-lactim pairs, enamine-imine pairs, and annular forms where a proton can occupy two or more positions of a heterocyclic system, for example, 1H- and 3H-imidazole, 1H-, 2H- and 4H-1,2,4-triazole, 1H- and 2H-isoindole, and 1H- and 2H-pyrazole. Tautomeric forms can be in equilibrium or sterically locked into one form by appropriate substitution.

Compounds described herein can also include isotopically-labeled compounds of the disclosure. An “isotopically” or “radio-labeled” compound is a compound of the disclosure where one or more atoms are replaced or substituted by an atom having an atomic mass or mass number different from the atomic mass or mass number typically found in nature (i.e., naturally occurring). Suitable radionuclides that may be incorporated in compounds of the present disclosure include but are not limited to ²H (also written as D for deuterium), ³H (also written as T for tritium), ¹¹C, ¹³C, ¹⁴C, ¹³N, ¹⁵N, ¹⁵O, ¹⁷O, ¹⁸O, ¹²F, ³³S, ³⁶Cl, ⁸²Br, ⁷⁵Br, ⁷⁶Br, ⁷⁷Br, ¹²³I, ¹²⁴I, ¹²⁵I and ¹³¹I. For example, one or more hydrogen atoms in a compound of the present disclosure can be replaced by deuterium atoms (e.g., one or more hydrogen atoms of a C₁₋₆ alkyl group of Formulae (I), (II), or (III) or a compound of Table 1 can be optionally substituted with deuterium atoms, such as —CD₃ being substituted for —CH₃). The term, “compound,” as used herein is meant to include all stereoisomers, geometric isomers, tautomers, and isotopes of the structures depicted, unless the name indicates a specific stereoisomer. Compounds herein identified by name or structure as one particular tautomeric form are intended to include other tautomeric forms unless otherwise specified.

All compounds, and pharmaceutically acceptable salts thereof, can be found together with other substances such as water and solvents (e.g. hydrates and solvates) or can be isolated.

In some embodiments, the compounds described herein, or salts thereof, are substantially isolated. By “substantially isolated” is meant that the compound is at least partially or substantially separated from the environment in which it was formed or detected. Partial separation can include, for example, a composition enriched in the compounds described herein. Substantial separation can include compositions containing at least about 50%, at least about 60%, at least about 70%, at least about 80%, at least about 90%, at least about 95%, at least about 97%, or at least about 99% by weight of the compounds described herein, or salt thereof. Methods for isolating compounds and their salts are routine in the art.

The phrase “pharmaceutically acceptable” is employed herein to refer to those compounds, materials, compositions, and/or dosage forms which are, within the scope of sound medical judgment, suitable for use in contact with the tissues of human beings and animals without excessive toxicity, irritation, allergic response, or other problem or complication, commensurate with a reasonable benefit/risk ratio.

The expressions, “ambient temperature” and “room temperature” or “rt” as used herein, are understood in the art, and refer generally to a temperature, e.g. a reaction temperature, that is about the temperature of the room in which the reaction is carried out, for example, a temperature from about 20° C. to about 30° C.

The present invention also includes pharmaceutically acceptable salts of the compounds described herein. As used herein, “pharmaceutically acceptable salts” refers to derivatives of the disclosed compounds wherein the parent compound is modified by converting an existing acid or base moiety to its salt form. Examples of pharmaceutically acceptable salts include, but are not limited to, mineral or organic acid salts of basic residues such as amines; alkali or organic salts of acidic residues such as carboxylic acids; and the like. The pharmaceutically acceptable salts of the present invention include the conventional non-toxic salts of the parent compound formed, for example, from non-toxic inorganic or organic acids. The pharmaceutically acceptable salts of the present invention can be synthesized from the parent compound which contains a basic or acidic moiety by conventional chemical methods. Generally, such salts can be prepared by reacting the free acid or base forms of these compounds with a stoichiometric amount of the appropriate base or acid in water or in an organic solvent, or in a mixture of the two; generally, non-aqueous media like ether, ethyl acetate, alcohols (e.g., methanol, ethanol, iso-propanol, or butanol) or acetonitrile (ACN) are preferred. Lists of suitable salts are found in Remington's Pharmaceutical Sciences, 17th ed., Mack Publishing Company, Easton, Pa., 1985, p. 1418 and Journal of Pharmaceutical Science, 66, 2 (1977), each of which is incorporated herein by reference in its entirety.

As used herein, the term “subject”, “individual,” or “patient,” used interchangeably, refers to any animal, including mammals, preferably mice, rats, other rodents, rabbits, dogs, cats, swine, cattle, sheep, horses, or primates, and most preferably humans In some embodiments, the “subject,” “individual,” or “patient” is in need of said treatment.

In some embodiments, the inhibitors are administered in a therapeutically effective amount. As used herein, the phrase “therapeutically effective amount” refers to the amount of active compound or pharmaceutical agent that elicits the biological or medicinal response that is being sought in a tissue, system, animal, individual or human by a researcher, veterinarian, medical doctor or other clinician.

As used herein, the term “treating” or “treatment” refers to one or more of (1) inhibiting the disease; for example, inhibiting a disease, condition or disorder in an individual who is experiencing or displaying the pathology or symptomatology of the disease, condition or disorder (i.e., arresting further development of the pathology and/or symptomatology); (2) ameliorating the disease; for example, ameliorating a disease, condition or disorder in an individual who is experiencing or displaying the pathology or symptomatology of the disease, condition or disorder (i.e., reversing the pathology and/or symptomatology) such as decreasing the severity of disease.

In some embodiments, the JAK1 inhibitors can prevent asthma in an individual who may be predisposed to the disease. The term “preventing” refers to blocking the occurrence of disease in a patient who may be predisposed to the disease but does not yet experience or display the pathology or symptomatology of the disease.

### Combination Therapies

The methods described herein can further comprise administering one or more additional therapeutic agents. The one or more additional therapeutic agents can be administered to a patient simultaneously or sequentially. The one or more additional therapeutic agents can be administered using different methods than Compound 1 (e.g., topically).

In some embodiments, the additional therapeutic agent is selected from other JAK inhibitors. Additional JAK inhibitors may include ATI-50002 (JAK1/3 selective). Additional JAK inhibitors may include PF-06651600 (JAK3 selective). Additional JAK inhibitors may include PF06700841 (JAK1/TYK2 selective). Additional JAK inhibitors may include Upadacitinib. Additional JAK inhibitors may include Abrocitinib (JAK1 selective). Additional JAK inhibitors may include Cerdulatinib (JAK1/SYK selective). Additional JAK inhibitors may include Deucravacitinib (TYK2 selective).

In some embodiments, the additional therapeutic agent is a medium-to-high dose inhaled corticosteroid (ICS). ICS agents may include beclomethasone, budesonide, budesonide/formoterol combination, fluticasone, fluticasone inh powder, fluticasone/salmeterol combination, mometasone, and/or mometasone/formoterol combination.

In some embodiments, the additional therapeutic agent is a long-acting bronchodilators (LABA). LABA agents may include salmeterol, formoterol, olodaterol, and/or theophylline. ICS and LABA may be used in combination.

In some embodiments, the additional therapeutic agent is a systemic oral corticosteroids (OCS). OCS agents may include betamethasone, budesonide, deflazacort, dexamethasone, dexamethasone sodium phosphate, fludrocortisone acetate, hydrocortisone, methylprednisolone acetate, prednisolone, and/or prednisolone.

In some embodiments, the additional therapeutic agent is a short-acting beta-agonist (SABA). SABA agents may include albuterol/salbutamol and/or levalbuterol.

In some embodiments, the additional therapeutic agent includes immunomodulators. Immunomodulators may include PDE4 inhibitors (e.g., apremilast (e.g., orally) or crisaborole (e.g., topically)). Immunomodulators may include anti-CD20 therapy (e g., ofatumumab) Immunomodulators may include anti-CD19 therapy (e.g., tafasitamab). Immunomodulators may include anti-IL15 therapy (e.g., AMG 714 monoclonal antibody). Immunomodulators may include anti-IL36 therapy (e.g., imsidolimab and spesolimab). Immunomodulators may include anti-TNFalpha therapy (e.g., etanercept and infliximab). Immunomodulators may include anti-CD122 therapy.

In some embodiments, immunomodulators are selected from apremilast, crisaborole, afamelanotide, rituximab, ofatumumab, tafasitamab, minocycline, latanoprost, zinc, tofacitinib, AMG 714 monoclonal antibodies, imsidolimab, spesolimabcyclosporine, etanercept, infliximab, cyclophosphamide, ciclosporin, methotrexate, and sodium oxo-dihydro-acridinylacetate (ODHAA).

In some embodiments, the additional therapeutic agent is a Janus kinase inhibitor. In some embodiments, the Janus kinase inhibitor is ruxolitinib, or a pharmaceutically acceptable salt thereof.

In some embodiments, the additional therapeutic agent is an IL-6 antagonist or receptor antagonist. In some embodiments, the IL-6 receptor antagonist is tocilizumab.

### Pharmaceutical Formidations and Dosage Forms

When employed as pharmaceuticals, the JAK1 pathway inhibitors or pharmaceutically acceptable salts thereof, can be administered in the form of pharmaceutical compositions. These compositions can be prepared in a manner well known in the pharmaceutical art, and can be administered by a variety of routes, depending upon whether local or systemic treatment is desired and upon the area to be treated. Administration may be topical (including transdermal, epidermal, ophthalmic and to mucous membranes including intranasal, vaginal and rectal delivery), pulmonary (e g., by inhalation or insufflation of powders or aerosols, including by nebulizer; intratracheal or intranasal), oral or parenteral. Parenteral administration includes intravenous, intraarterial, subcutaneous, intraperitoneal intramuscular or injection or infusion; or intracranial, e.g., intrathecal or intraventricular, administration. Parenteral administration can be in the form of a single bolus dose, or may be, for example, by a continuous perfusion pump. Pharmaceutical compositions and formulations for topical administration may include transdermal patches, ointments, lotions, creams, gels, drops, suppositories, sprays, foams, liquids and powders. Conventional pharmaceutical carriers, aqueous, powder or oily bases, thickeners and the like may be necessary or desirable.

Pharmaceutical compositions and formulations for may be administered orally. Oral administration may include using tablets. The composition may be administered orally in a tablet with water.

In some embodiments, a JAK1 pathway inhibitor, or a pharmaceutically acceptable salt thereof, is used for manufacture of a medicament. The medicament may be used for treating asthma in a subject in need thereof. The medicament may be used for treating eosinophilic asthma in a subject in need thereof. The medicament may be used for treating noneosinophilic asthma in a subject in need thereof.

This invention also includes pharmaceutical compositions which contain, as the active ingredient, the JAK1 pathway inhibitor described herein, or a pharmaceutically acceptable salt thereof, in combination with one or more pharmaceutically acceptable carriers (excipients). In some embodiments, the composition is suitable for topical administration. In making the compositions, the active ingredient is typically mixed with an excipient, diluted by an excipient or enclosed within such a carrier in the form of, for example, a capsule, sachet, paper, or other container. When the excipient serves as a diluent, it can be a solid, semi-solid, or liquid material, which acts as a vehicle, carrier or medium for the active ingredient. Thus, the compositions can be in the form of tablets, pills, powders, lozenges, sachets, cachets, elixirs, suspensions, emulsions, solutions, syrups, aerosols (as a solid or in a liquid medium), ointments containing, for example, up to 10% by weight of the active compound, soft and hard gelatin capsules, suppositories, sterile injectable solutions, and sterile packaged powders.

In preparing a formulation, the active compound can be milled to provide the appropriate particle size prior to combining with the other ingredients. If the active compound is substantially insoluble, it can be milled to a particle size of less than 200 mesh. If the active compound is substantially water soluble, the particle size can be adjusted by milling to provide a substantially uniform distribution in the formulation, e.g., about 40 mesh.

The JAK1 pathway inhibitors may be milled using known milling procedures such as wet milling to obtain a particle size appropriate for tablet formation and for other formulation types. Finely divided (nanoparticulate) preparations of the JAK1 selective inhibitors can be prepared by processes known in the art, e.g., see International App. No. WO 2002/000196.

The compositions can be formulated in a unit dosage form, each dosage containing a set amount of the active ingredient as the free form or a salt form. The term “unit dosage forms” refers to physically discrete units suitable as unitary dosages for human subjects and other mammals, each unit containing a predetermined quantity of active material calculated to produce the desired therapeutic effect, in association with a suitable pharmaceutical excipient.

Similar dosages may be used of the compounds described herein in the methods and uses of the invention.

The active compound can be effective over a wide dosage range and is generally administered in a pharmaceutically effective amount. It will be understood, however, that the amount of the compound actually administered will usually be determined by a physician, according to the relevant circumstances, including the condition to be treated, the chosen route of administration, the actual compound administered, the age, weight, and response of the individual patient, the severity of the patient's symptoms, and the like.

For preparing solid compositions such as tablets, the principal active ingredient is mixed with a pharmaceutical excipient to form a solid preformulation composition containing a homogeneous mixture of a compound of the present invention. When referring to these preformulation compositions as homogeneous, the active ingredient is typically dispersed evenly throughout the composition so that the composition can be readily subdivided into equally effective unit dosage forms such as tablets, pills and capsules. This solid preformulation is then subdivided into unit dosage forms of the type described above containing from, for example, about 0.1 to about 1000 mg of the active ingredient of the present invention.

The tablets or pills of the present invention can be coated or otherwise compounded to provide a dosage form affording the advantage of prolonged action. For example, the tablet or pill can comprise an inner dosage and an outer dosage component, the latter being in the form of an envelope over the former. The two components can be separated by an enteric layer which serves to resist disintegration in the stomach and permit the inner component to pass intact into the duodenum or to be delayed in release. A variety of materials can be used for such enteric layers or coatings, such materials including a number of polymeric acids and mixtures of polymeric acids with such materials as shellac, cetyl alcohol, and cellulose acetate.

The liquid forms in which the compounds and compositions of the present invention can be incorporated for administration orally or by injection include aqueous solutions, suitably flavored syrups, aqueous or oil suspensions, and flavored emulsions with edible oils such as cottonseed oil, sesame oil, coconut oil, or peanut oil, as well as elixirs and similar pharmaceutical vehicles.

Compositions for inhalation or insufflation include solutions and suspensions in pharmaceutically acceptable, aqueous or organic solvents, or mixtures thereof, and powders. The liquid or solid compositions may contain suitable pharmaceutically acceptable excipients as described supra. In some embodiments, the compositions are administered by the oral or nasal respiratory route for local or systemic effect. Compositions can be nebulized by use of inert gases. Nebulized solutions may be breathed directly from the nebulizing device or the nebulizing device can be attached to a face mask, tent, or intermittent positive pressure breathing machine. Solution, suspension, or powder compositions can be administered orally or nasally from devices which deliver the formulation in an appropriate manner.

Topical formulations can contain one or more conventional carriers. In some embodiments, ointments can contain water and one or more hydrophobic carriers selected from, for example, liquid paraffin, polyoxyethylene alkyl ether, propylene glycol, white Vaseline, and the like. Carrier compositions of creams can be based on water in combination with glycerol and one or more other components, e.g., glycerinemonostearate, PEG-glycerinemonostearate and cetylstearyl alcohol. Gels can be formulated using isopropyl alcohol and water, suitably in combination with other components such as, for example, glycerol, hydroxyethyl cellulose, and the like.

The amount of compound or composition administered to a patient will vary depending upon what is being administered, the purpose of the administration, such as prophylaxis or therapy, the state of the patient, the manner of administration, and the like. In therapeutic applications, compositions can be administered to a patient already suffering from a disease in an amount sufficient to cure or at least partially arrest the symptoms of the disease and its complications. Effective doses will depend on the disease condition being treated as well as by the judgment of the attending clinician depending upon factors such as the severity of the disease, the age, weight and general condition of the patient, and the like.

The compositions administered to a patient can be in the form of pharmaceutical compositions described above. These compositions can be sterilized by conventional sterilization techniques, or may be sterile filtered. Aqueous solutions can be packaged for use as is, or lyophilized, the lyophilized preparation being combined with a sterile aqueous carrier prior to administration The pH of the compound preparations typically will be between 3 and 11, more preferably from 5 to 9 and most preferably from 7 to 8. It will be understood that use of certain of the foregoing excipients, carriers, or stabilizers will result in the formation of pharmaceutical salts.

The therapeutic dosage of a compound of the present invention can vary according to, for example, the particular use for which the treatment is made, the manner of administration of the compound, the health and condition of the patient, and the judgment of the prescribing physician. The proportion or concentration of a compound described herein in a pharmaceutical composition can vary depending upon a number of factors including dosage, chemical characteristics (e.g., hydrophobicity), and the route of administration. The dosage is likely to depend on such variables as the type and extent of progression of the disease or disorder, the overall health status of the particular patient, the relative biological efficacy of the compound selected, formulation of the excipient, and its route of administration. Effective doses can be extrapolated from dose-response curves derived from in vitro or animal model test systems.

The compositions of the invention can further include one or more additional pharmaceutical agents such as a chemotherapeutic, steroid, anti-inflammatory compound, or immunosuppressant, examples of which are listed herein.

### Kits

The present invention also includes pharmaceutical kits useful, for example, in the treatment and/or prevention of asthma, which include one or more containers containing a pharmaceutical composition comprising a therapeutically effective amount of a compound described herein. Such kits can further include, if desired, one or more of various conventional pharmaceutical kit components, such as, for example, containers with one or more pharmaceutically acceptable carriers, additional containers, nebulizers, etc., as will be readily apparent to those skilled in the art. Instructions, either as inserts or as labels, indicating quantities of the components to be administered, guidelines for administration, and/or guidelines for mixing the components, can also be included in the kit

### EXAMPLES

The invention will be described in greater detail by way of specific examples. The following examples are offered for illustrative purposes, and are not intended to limit the invention in any manner. Those of skill in the art will readily recognize a variety of non-critical parameters which can be changed or modified to yield essentially the same results. The compounds of the Examples have been found to be JAK inhibitors according to at least one assay described herein.

### Example A: In Vitro JAK Kinase Assay

JAK1 pathway inhibitors that can be used for the treatment of cytokine-related diseases or disorders were tested for inhibitory activity of JAK targets according to the following in vitro assay described in Park et al., Analytical Biochemistry 1999, 269, 94-104. The catalytic domains of human JAK1 (a.a. 837-1142), JAK2 (a.a. 828-1132) and JAK3 (a.a. 781-1124) with an N-terminal His tag were expressed using baculovirus in insect cells and purified. The catalytic activity of JAK1, JAK2 or JAK3 was assayed by measuring the phosphorylation of a biotinylated peptide. The phosphorylated peptide was detected by homogenous time resolved fluorescence (HTRF). IC₅₀s of compounds were measured for each kinase in the 40 microL reactions that contain the enzyme, ATP and 500 nM peptide in 50 mM Tris (pH 7.8) buffer with 100 mM NaCl, 5 mM DTT, and 0.1 mg/mL (0.01%) BSA. For the 1 mM IC₅₀ measurements, ATP concentration in the reactions is 1 mM. Reactions were carried out at room temperature for 1 hour and then stopped with 20 μL 45 mM EDTA, 300 nM SA-APC, 6 nM Eu-Py20 in assay buffer (Perkin Elmer, Boston, MA). Binding to the Europium labeled antibody took place for 40 minutes and HTRF signal was measured on a Fusion plate reader (Perkin Elmer, Boston, MA). The compounds in Table 1 were tested in this assay and shown to have the IC₅₀ values also found in Table 1.

### Example B: Proposed Phase 2 Study of Compound 1

### Study Design

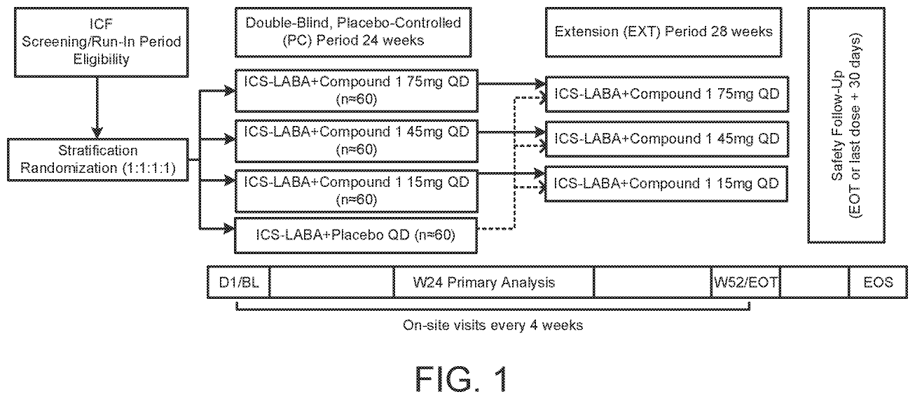

A Phase 2 study may include a double-blind, placebo-controlled, multicenter study of Compound 1 on stable background therapy with medium-to-high dose inhaled corticosteroids in combination with a long-acting bronchodilator (ICS-LABA). FIG. 1 depicts an outline of a phase 2 randomized, double-blind, placebo-controlled dose-ranging study of the efficacy and safety of Compound 1. The study will enroll participants (e.g., about 240) in a 1:1:1:1 randomization ratio. The participants may be stratified by eosinophil count at screening (≥150 cells/μL vs <150 cells/μL). In some embodiments, a JAK1 inhibitor, e.g., Compound 1, and/or methods of use described herein are effective for treating subjects with an eosinophil count greater than or equal to 150 cell/μL. In some embodiments, a JAK1 inhibitor, e.g., Compound 1, and/or methods of use described herein are effective for treating subjects with an eosinophil count less than 150 cell/μL. The participants may be stratified by previous treatment of asthma with a biologic agent (yes or no). The participants may be stratified into 1 of 4 treatment groups during the 24-week placebo controlled (PC) period:
ICS-LABA+Compound 1 75 mg QD (n≈60)
ICS-LABA+Compound 1 45 mg QD (n≈60)
ICS-LABA+Compound 1 15 mg QD (n≈60)
ICS-LABA+placebo QD (n≈60).

In some embodiments, the study will include up to 28 days for screening/run-in, continuous treatment for 52 weeks (including the PC and extension (EXT) periods), and 30 (+7) days for safety follow-up after the last dose of study drug. It is estimated that an individual will participate for approximately 14 months

After completing the 24-week PC period, participants will enter the 28-week EXT period. During the EXT period, the participants initially randomized to Compound 1 (e.g., Compound 1) Dose A, Dose B, and Dose C will continue taking Compound 1 at the same dose. The participants initially randomized to placebo will be equally allocated to take 1 of these 3 doses of Compound 1. Participants and investigators will remain blinded to Compound 1 dose as well as prior treatment assignment during the EXT period.

Participants will receive study drug until completion of the 52-week treatment period or until one of the criteria for study treatment discontinuation are met. Participants will return for a safety follow-up visit approximately 30 days after their last dose of study drug.

All participants may be required to be treated with a stable dose of medium-to high-dose ICS-LABA during the study (i.e., during the screening/run-in, PC, and EXT periods and until completion of the safety follow-up visit [EOS]). Additionally, participants may use rescue medication (SABA or SMART) to treat worsening asthma symptoms during the study.

Participants will undergo regular efficacy assessments throughout the study. Efficacy may be evaluated by spirometry and assessment of asthma exacerbations. Fractional exhaled nitric oxide may be used as a measure of airway inflammation. The participant may perform PEF testing BID at home. Questionnaires may be used to evaluate the impact of treatment on asthma symptoms and health related QoL.

Participants may complete an eDiary BID from the start of the run-in period (Visit 2) until the completion of the safety follow-up visit (EOS). The eDiary may provide data on the participant's asthma symptoms and nighttime awakenings and use of rescue medication.

Serum samples may be collected for biomarker/translational analysis, and blood samples will be collected throughout the study for measurement of systemic concentrations of Compound 1.

The primary efficacy analysis of change from baseline in pre-BD FEV1 may be performed after all participants have either completed the Week 24 visit or discontinued from the study. Treatment assignments will be unblinded to the sponsor at the time of the Week 24 primary analysis; however, the double-blind will be maintained for both the participant and site staff throughout the study.

In some embodiments, endpoints of the study include absolute change from baseline in pre-BD FEV₁ (e g., at week 24) as regards the effect of Compound 1 on pulmonary function. Endpoints of the study include number of asthma exacerbations during (defined as a worsening of asthma) as regards the effect of Compound 1 on asthma exacerbations. In some embodiments, a JAK1 inhibitor, e.g., Compound 1, and/or methods of use described herein may result in about a 5%, about a 10%, about a 20%, about a 30%, about a 40%, about a 50%, about a 60%, about a 70%, about a 80%, about a 90%, or about a 95% improvement in study endpoints. In some embodiments, a JAK1 inhibitor, e.g., Compound 1, and/or methods of use described herein may result in about a 5%, about a 10%, about a 20%, about a 30%, about a 40%, about a 50%, about a 60%, about a 70%, about a 80%, about a 90%, or about a 95% decrease in asthma exacerbations.

In some embodiments, an objective is to determine the safety of Compound 1. In some embodiments, an endpoint of the study includes absolute and percent change from baseline in FeNO at each visit as regards effect of Compound 1 on airway inflammation. In some embodiments, an endpoint of the study includes change from baseline in AQLQ(S) score, EQ-5D-5L score, and/or WPAI:Asthma score at each visit as regards effect of Compound 1 on asthma-related and general health-related QoL. In some embodiments, an endpoint of the study includes change from baseline in ACQ-6 score, ACQ-5 score, PGI-S score, ASD score, rescue medication (SABA or SMART) use, and/or Home lung function as regards effect of Compound 1 on asthma symptoms and other asthma control metrics. In some embodiments, an endpoint of the study includes score of CGI-C and/or PGI-C as regards effect of Compound 1 on the overall impression of treatment of asthma. In some embodiments, an endpoint of the study includes change from baseline in SNOT-22 score as regards effect of Compound 1 on chronic rhinosinusitis/nasal polyposis symptoms in participants with a medical history of current/ongoing chronic rhinosinusitis and/or bilateral nasal polyposis. In some embodiments, an endpoint of the study includes expression of select biomarkers in peripheral blood at baseline as regards effect of Compound 1 to explore blood biomarkers in participants. In some embodiments, an endpoint of the study includes Population PK parameters of Compound 1 such as apparent clearance, apparent volume of distribution, apparent oral absorption rate constant, and absorption lag time, as deemed applicable, as well as model-based post hoc predictions of steady-state PK exposures such as C_{max,ss}, C_{avg,ss}, C_{tau,ss}, t_{max,ss}, and t_{1/2} as regards effect of Compound 1 to determine the systemic exposure. In some embodiments, an endpoint of the study includes population PK/PD analysis of select clinical response endpoints as regards effect of Compound 1 to determine the Compound 1 PK/PD relationship.

Participants may be eligible to be included in the study if male or female and 18 to 65 years of age. Participants may be eligible to be included in the study if the participant has physician-diagnosed asthma requiring treatment with medium-to high-dose ICS-LABA for at least 12 months prior to screening. Participants may be eligible to be included in the study if the participant has documented treatment with a stable daily dose of medium-dose ICS (e.g., ≥250 μg fluticasone dry powder formulation equivalents total daily dose) or high-dose ICS (e.g., >500 μg fluticasone dry powder formulation equivalents total daily dose) and a LABA for at least 3 months prior to screening. Participants may be eligible to be included in the study if the participant agrees to use a medium-to high-dose ICS-LABA at a stable dose from screening through the duration of the study. Participants may be eligible to be included in the study if the participant has pre-BD FEV₁<80% predicted according to central overread value at Visit 2. Participants may be eligible to be included in the study if the participant has documented historical post-BD reversibility of FEV₁≥12% and ≥200 mL in FEV₁ within 12 months prior to screening. Participants may be eligible to be included in the study if the participant has documented historical post-BD reversibility of FEV₁≥12% and ≥200 mL in FEV₁ according to central overread value at Visit 2. Participants may be eligible to be included in the study if the participant has at least 2 documented asthma exacerbations (e.g., requiring treatment with systemic CS, hospitalization, or emergency department visit) within 12 months prior to screening but not within the past 4 weeks prior to screening. Participants may be eligible to be included in the study if the participant has ACQ-6≥1.5 at screening. Participants may be eligible to be included in the study if the participant agrees to use contraception.

In some embodiments, a JAK1 inhibitor, e.g., Compound 1, and/or methods of use described herein may result in an improvement in the number of asthma exacerbations during the PC period.

In some embodiments, a JAK1 inhibitor, e.g., Compound 1, and/or methods of use described herein may result in a reduction in the use of systemic CS for consecutive days, such as 2 days, 3 days, 4 days, or 5 days. In some embodiments, a single depo-injectable dose of CS will be considered equivalent to a 3-day course of systemic CS.

In some embodiments, a JAK1 inhibitor, e.g., Compound 1, and/or methods of use described herein may result in a reduction of emergency department or urgent care visits (defined as evaluation and treatment for <24 hours in an emergency department or urgent care center) due to asthma that required use of systemic CS. In some embodiments, a JAK1 inhibitor, e.g., Compound 1, and/or methods of use described herein may result in a reduction of an inpatient hospitalization (defined as admission to an inpatient facility and/or evaluation and treatment in a healthcare facility for ≥24 hours) due to asthma.

In some embodiments, criteria for participants to be excluded from the study include experiencing 1 or more asthma exacerbation during screening/run-in. Participants may be excluded for an inability to use acceptable inhaler technique or to perform PEF and spirometry assessments. Participants may be excluded for an inability to receive or initiation of medical treatment or procedures used for asthma management within a prescribed period. Participants may be excluded for having undergone bronchial thermoplasty. Participants may be excluded for being current smokers (e.g., tobacco, vaping products, electronic cigarettes) or participants with a smoking history of ≥10 pack-years. Participants may be excluded for being pregnant (or who are considering pregnancy) or breastfeeding. Participants may be excluded for one or more, or two or more, or three or more, certain current conditions or history of other diseases, as follows:
a) Clinically important pulmonary disease other than asthma (eg, active lung infection, chronic obstructive pulmonary disease, bronchiectasis, pulmonary fibrosis, cystic fibrosis, hypoventilation syndrome associated with obesity, lung cancer, alpha-1 antitrypsin deficiency, primary ciliary dyskinesia, allergic bronchopulmonary aspergillosis/mycosis, Churg-Strauss syndrome, and hypereosinophilic syndrome);
b) Thrombocytopenia, coagulopathy, or platelet dysfunction;
c) Venous and arterial thrombosis, deep vein thrombosis, pulmonary embolism, moderate to severe heart failure (NYHA Class III or IV), cerebrovascular accident, myocardial infarction, coronary stenting, or CABG surgery;
d) Diagnosis of other significant cardiovascular diseases, including but not limited to angina, peripheral arterial disease, or uncontrolled arrhythmias such as atrial fibrillation, supraventricular tachycardia, ventricular tachycardia, and forms of carditis;
e) Uncontrolled hypertension, as defined by a confirmed systolic blood pressure >160 mm Hg or diastolic blood pressure >100 mm Hg;
f) Permanently bedridden or wheelchair assisted;
g) Recipient of an organ transplant that requires continued immunosuppression;
h) Immunocompromised (eg, lymphoma, acquired immunodeficiency syndrome, Wiskott-Aldrich syndrome);
i) Any malignancies or history of malignancies.
Note: Participants with cured nonmetastatic basal cell or squamous cell carcinoma of the skin, superficial bladder cancer, prostate intraepithelial neoplasm, carcinoma in situ of the cervix, or other noninvasive malignancy or cancers from which the participant has been disease-free for >1 year after treatment with curative intent are eligible.
j) Conditions that could interfere with drug absorption, including but not limited to short-bowel syndrome.
k) Chronic or recurrent infectious disease, including but not limited to chronic renal infection, chronic chest infection (eg, bronchiectasis), recurrent urinary tract infection (recurrent pyelonephritis or chronic nonremitting cystitis), fungal infection, prior prosthetic joint infection at any time, or open, draining, or infected skin wounds or ulcers.
l) Current or history of disseminated herpes zoster, or recurrent (more than 1 episode of) dermatomal herpes zoster.
m) Current or history of disseminated herpes simplex.
n) Active systemic infection or any active infection that, based on the investigator's clinical assessment, makes the participant an unsuitable candidate for the study.
o) Any clinically significant medical condition (other than asthma) or any other reason that the investigator determines would interfere with the participant's participation in this study or would make the participant an unsuitable candidate to receive study drug or would put the participant at risk by participating in the study.
p) Any clinically significant medical condition other than asthma, as determined by the investigator, that is not adequately controlled with appropriate treatment or may interfere with the course, severity, or assessments of asthma.
q) Albinism.

Participants may be excluded for having acute upper or lower respiratory infections requiring antibiotics or antiviral medication. Participants may be excluded for having a screening 12-lead ECG that demonstrates clinically significant abnormalities requiring treatment. Participants may be excluded for having undergone significant trauma or major surgery (previous or planned). Participants may be excluded for having history of clinically significant drug or alcohol abuse. Participants may be excluded for having history of treatment failure with any topical or systemic JAK inhibitor for any inflammatory condition, including asthma. Participants may be excluded for having received medical treatment or investigational drugs within a certain time period dependent on the treatment/drugs. Participants may be excluded for having concurrent enrollment in another clinical study. Participants may be excluded for having any laboratory abnormalities. Participants may be excluded for having Evidence of infection with Mycobacterium tuberculosis (i.e., TB). Participants may be excluded for having active HIV or acquired immunodeficiency syndrome. Active HIV is defined as a confirmed positive anti-HIV antibody test. Participants may be excluded for having evidence of HBV or HCV infection or risk of reactivation. Participants may be excluded for having known hypersensitivity or severe reaction to Compound 1 or excipients of Compound 1 and/or other products in the same class. Participants may be excluded for having

In some embodiments, criteria for participants to be excluded from the study include participants with one or more, or two or more, or three or more, of the laboratory values at screening defined in Table 2:

TABLE 2

| | |
|---|---|
| Laboratory Parameter | Exclusion Criterion |

Hematology

| | | |
|---|---|---|
| a | Platelets | <100 × 10⁹/L |
| b | Hemoglobin | <10 g/dL |
| c | ANC | <1.5 × 10⁹/L |
| d | Total white blood cell | <3.0 × 10⁹/L |
| | count (leukocyte count) | |
| e | Absolute lymphocyte count | <0.8 × 10⁹/L |

Hepatic

| | | |
|---|---|---|
| f | ALT | >2 × ULN |
| g | AST | >2 × ULN |
| h | Total bilirubin | >1.5 × ULN |
| | | (Note: Participants with clinical |
| | | diagnosis of Gilbert syndrome may |
| | | have a direct bilirubin measured |
| | | and would be eligible provided the |
| | | direct bilirubin is less than the ULN) |

Renal

| | | |
|---|---|---|
| i | Estimated glomerular | <45 mL/min per 1.73 m² |
| | filtration rate | Note: Based on the simplified |
| | | 4-variable MDRD formula |

### Example C: In Vitro Study of Compound 1

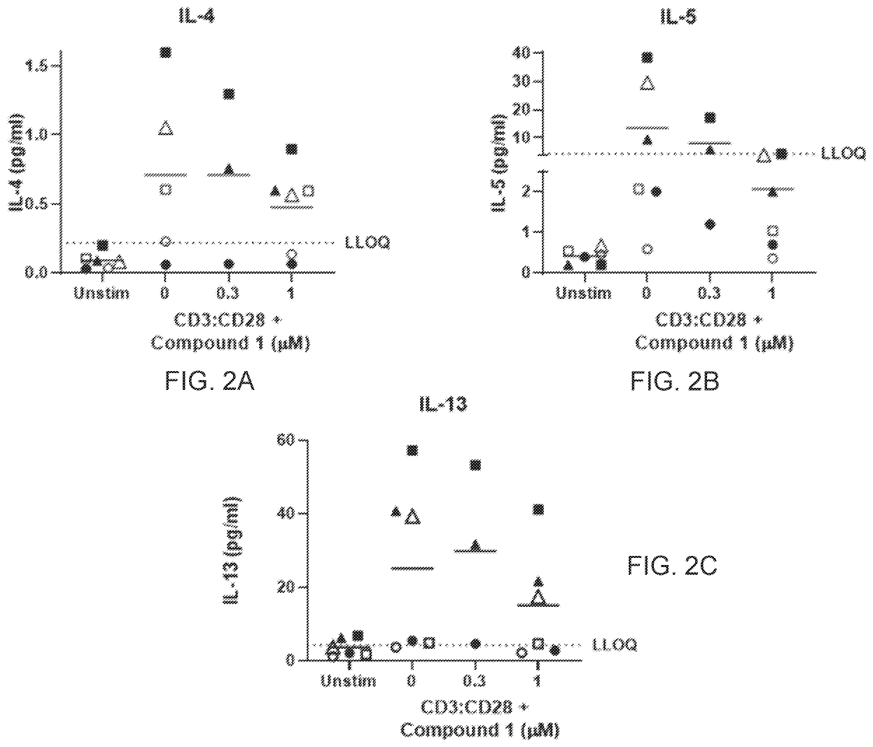

An in vitro study was conducted to investigate the impact of Compound 1 on eosinophil and neutrophil activation and cytokine production that play a role in contributing to eosinophilic and noneosinophilic (neutrophilic) asthma endotypes. Whole blood from healthy volunteers was unstimulated (Unstim) or stimulated with Dynabeads™ Human T-Activator CD3/CD28 in the absence (0) or presence (0.3 μM, 1 μM) of Compound 1 for a total of 19 hours at 37° C. Whole blood was analyzed for cell surface activation marker CD69 gated for eosinophils or neutrophils by flow cytometry (n=12). Plasma was isolated from the whole blood and analyzed for various cytokines using a MSD multiplex platform (n=6). As depicted in FIGS. 2-5, the presence of Compound 1 resulted in a concentration dependent reduction in cytokines and a reduction in eosinophil and neutrophil activation. Compound 1 significantly inhibits eosinophil and neutrophil activation by about 40%. Because Compound 1 inhibits the production of Th2/Th1/Th17 cytokines and reduces activation of eosinophils and neutrophils Compound 1 provides an endotype-agnostic means to treat severe asthma.

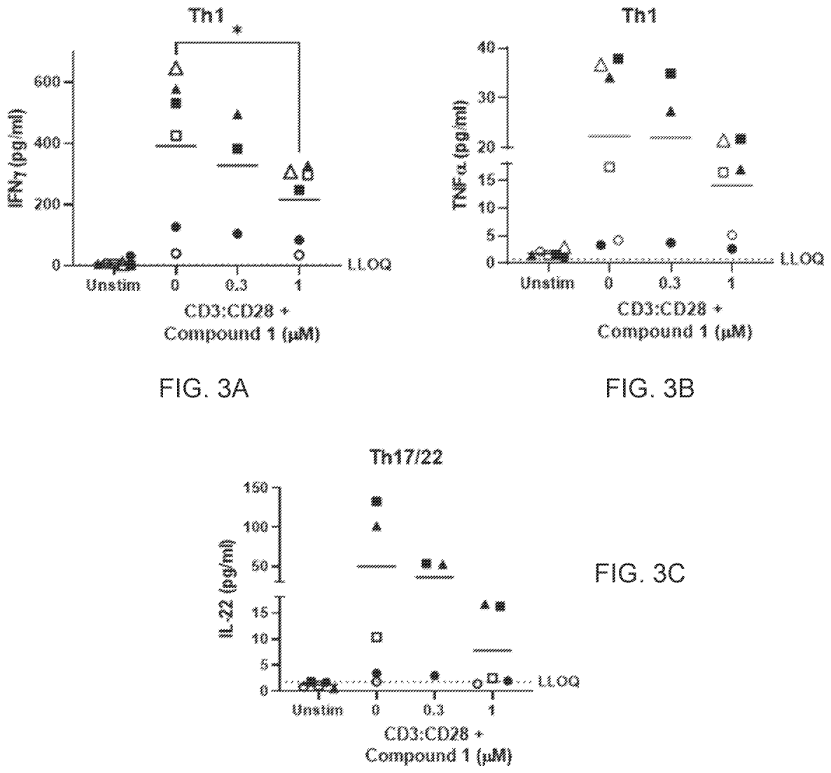

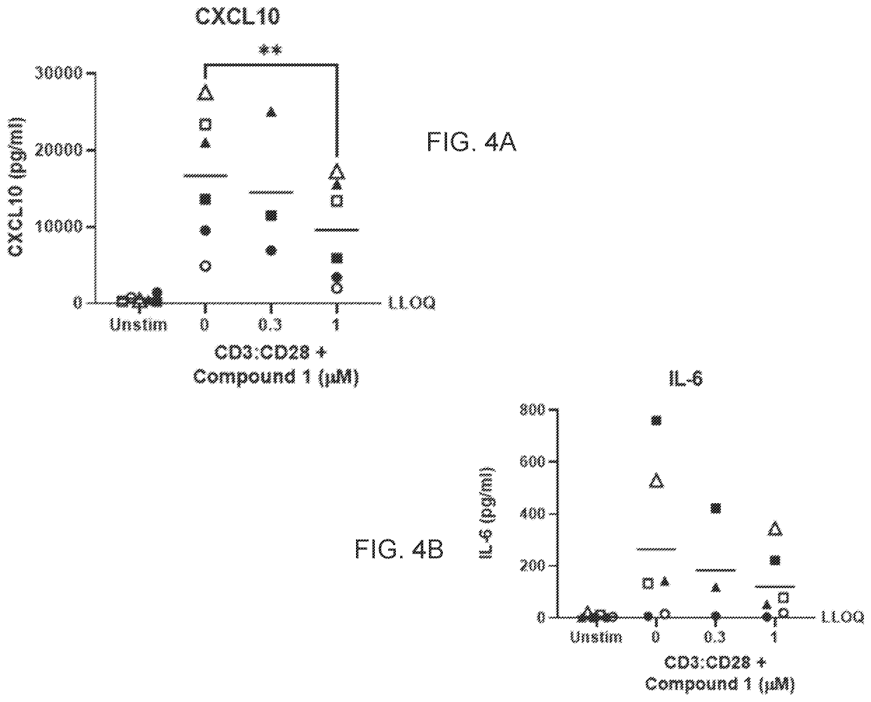

FIGS. 2-4 depict the effect of Compound 1 on cytokine levels in a whole blood assay. Plasma was isolated from whole blood samples and analyzed for Th2 cytokines (FIGS. 2A-C: IL-4, IL-5, and IL-13 respectively); Th1/Th17 cytokines (FIGS. 3A-C: IFNγ, TNFα, and IL-22 respectively), and other inflammatory cytokines/chemokines (FIGS. 4A-B: CXCL10 and IL-6 respectively). Each symbol represents a different donor. Values from individual donors are shown, along with mean value for each treatment. Data was plotted in GraphPad PRISM v.9.3.1 and analyzed by a paired t test (*:p<0.05; **:p≤0.01; ***:p≤0.001).

Donors include Donor 17 ▪; Donor 19 ●; Donor 20 ▴; Donor 36 ▾; Donor 37 ♦; Donor 38 [symbol] ; Donor 39 ⊙; Donor 40 ⊗; Donor 41 □; Donor 42 O, Donor 43 ∇; and Donor 50 Δ. FIGS. 2A-C show that Compound 1 reduced the levels of Th2 cytokines induced by T-cell activation in a concentration-dependent manner. FIGS. 3A-C show that Compound 1 reduced Th1 and Th17/Th22 cytokines induced by T-cell activation in a concentration-dependent manner. FIGS. 4A-B show that Compound 1 reduced inflammatory cytokines/chemokines induced by T cell activation in a concentration-dependent manner.

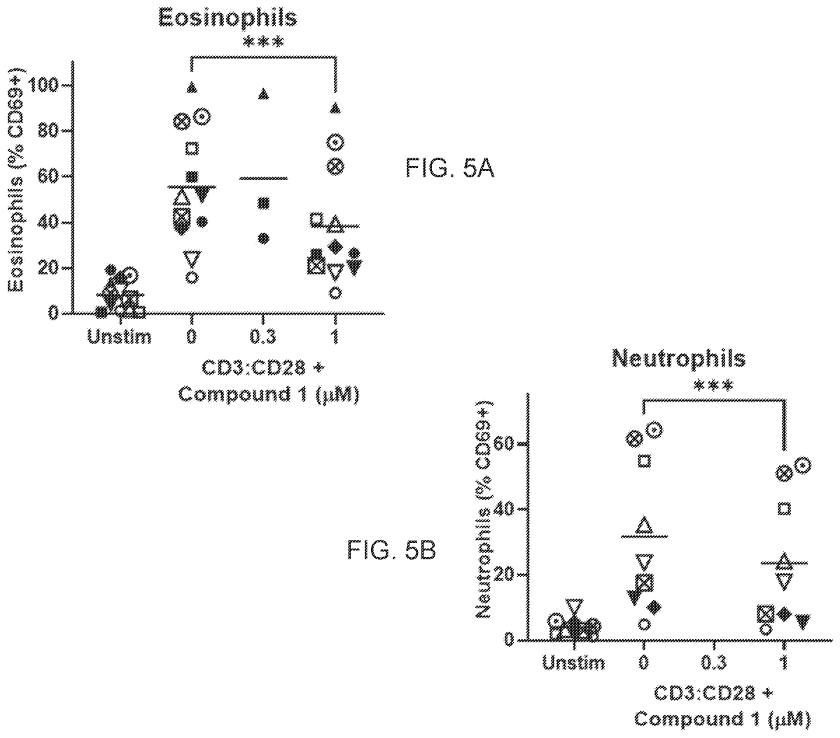

FIGS. 5A-B. Effect of Compound 1 on eosinophil and neutrophil activation marker CD69 in a whole blood assay. Each symbol represents a different donor. Values from individual donors are shown, along with mean value for each treatment. Data was plotted in GraphPad PRISM v.9.3.1 and analyzed by paired t test (*:p<0.05; **:p≤0.01; ***:p≤0.001). FIGS. 5A-B show that Compound 1 significantly reduced eosinophil and neutrophil activation marker CD69 by 40%. Together, these data show that unlike existing treatment options that exclusively target eosinophilic asthma, Compound 1 is effective in treating both eosinophilic and noneosinophilic (neutrophilic) asthma endotypes.

### Example D: Effects of Compound 1 Tested in Mouse Models of Severe Asthma

In vivo studies were conducted to test Compound 1 in two models of severe asthma. The first study conducted included an experimental house dust mite (HDM)-induced severe asthma model representative of both Th2/eosinophilic and Th1/Th17/neutrophilic asthma endotypes. The second study conducted included an ovalbumin (OVA) model of acute asthma which more closely represents a Th2/eosinophilic asthma endotype. For each model, fifty-eight female BALB/c mice were randomly and prospectively assigned to six groups, one group of six, one group of twelve, and four groups of ten animals each (see Table 3).

TABLE 3
Treatment groups in HDM and OVA models

| | No. | | | |
|---|---|---|---|---|
| Group | Animals | Treatment | Dose | |
| 1 | 6 | — | — | |
| 2 | 12 | Vehicle | — | |
| 3 | 10* | Compound 1 | 1 | mg/kg |
| 4 | 10 | | 10 | mg/kg |
| 5 | 10 | | 30 | mg/kg |
| 6 | 10 | Positive | 5 | mg/kg |
| | | Control | | |

*mice lost in HDM model due to dosing error.

HDM model: On Day 0, animals in Groups 2-6 were sensitized with 50 μg of HDM in an emulsion with 150 μg of complete Freund's adjuvant (CFA) in a 100 μL volume by subcutaneous injection (SC). Animals in Groups 2-6 were challenged on Day 14 with 50 μg HDM in 40 μL saline via intra-nasal instillation (IN). Group 1 served as the naïve control group. On Days 12, 13, 14, and 15, animals in Groups 2-6 were dosed according to Table 3. Due to dosing error, group 3 mice did not survive. On Day 16, animals underwent methacholine challenge with lung mechanics measurements using a flexi Vent mechanical ventilator.

OVA model: On Day 0 and Day 7, animals in Groups 2-6 were sensitized with 20 μg of OVA and 1.5 mg Alum in 100 μL volume by intraperitoneal injection (IP). Animals in Groups 2-6 were challenged on Day 13, 14, and 15 with 20 μg OVA in 50 μL PBS via intra-nasal instillation (IN). Group I served as the naïve control group. On Days 12, 13, 14, and 15, animals in Groups 2-6 were dosed according to Table 3. On Day 16, animals underwent methacholine challenge with lung mechanics measurements using a flexiVent mechanical ventilator.

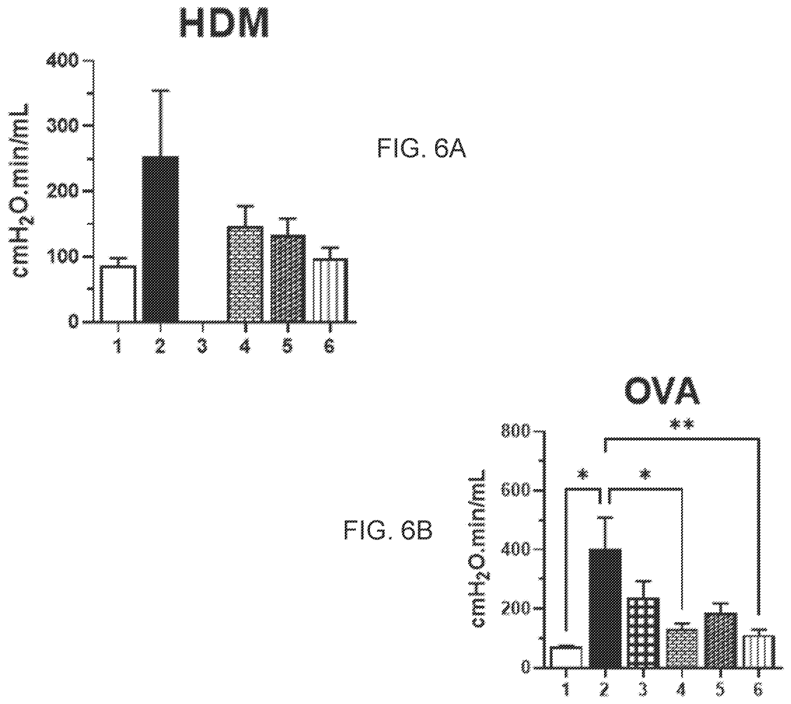

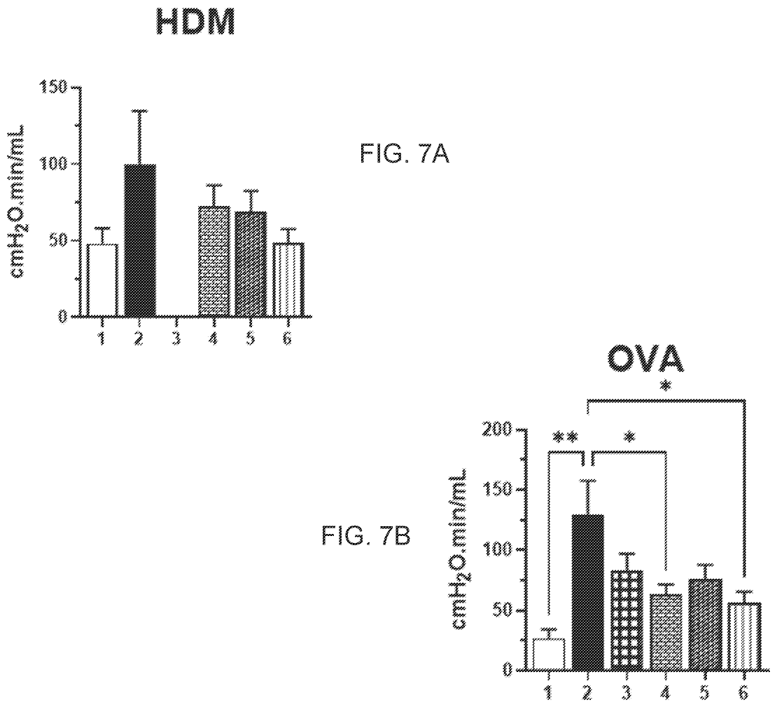

In the HDM model, measures of lung function following methacholine challenge indicated that diseased, vehicle treated animals had notably higher lung constriction (Rrs), higher pressure to inflate lungs (Ers), higher resistance in small airways and alveoli (G) and higher elasticity of the lung (H) in comparison to naïve animals. While no significant differences in airway measures were observed between groups including the positive control, there were decreases in a number of these measures, most notably in resistance in small airways and alveoli (G) and elasticity of the lung (H) in response to Compound 1, suggesting an improvement in lung function to treatment with Compound 1 in a mouse model representing both Th2/eosinophilic and Th1/Th17/neutrophilic asthma endotypes (FIGS. 6-7).

In the OVA model, measures of lung function following methacholine challenge indicated that diseased, vehicle treated animals had notably higher lung constriction (Rrs), higher pressure to inflate lungs (Ers), significantly higher resistance in small airways and alveoli (G) and significantly higher elasticity of the lung (H) in comparison to naïve animals. Compound 1 reversed the elevation of resistance in small airways and alveoli (G) and elasticity of the lung (H), suggesting an improvement in lung function to treatment with Compound 1 (FIGS. 6-7). A significant reduction in these parameters was observed with the 10 mg/kg dose. These data suggest that Compound 1 provides an improvement of lung function in a mouse model representing Th2/eosinophilic asthma.

FIGS. 6-7 show the effect of Compound 1 on lung function. Elasticity of the lung (H) shown in FIGS. 6A-B and airway resistance in small airways and alveoli (G) shown in FIGS. 7A-B were measured using a flexiVent mechanical ventilator following challenge with 25 mg/mL methacholine. Group numbers represent the following treatments: 1) naïve; 2) diseased, vehicle treated; 3) diseased, Compound 1 (1 mg/kg); 4) diseased, Compound 1 (10 mg/kg); 5) diseased, Compound 1 (30 mg/kg); and 6) diseased, positive control. Data are presented as mean±SEM. n=6-12 per group. Statistical significance between groups was determined by one-way ANOVA with Dunnett's multiple comparisons test used to compare all groups to the vehicle-control group. (*:p<0.05; **:p≤0.01).

Data from an OVA model of acute asthma and an HDM model of severe asthma, each of which demonstrate the hallmarks of both human eosinophilic and noneosinophilic (neutrophilic) asthma endotypes indicate that an orally bioavailable small molecule, Compound 1, is effective in reversing the disease in both models based on improvement of lung function. Together, these data demonstrate that unlike existing treatments including biologics that exclusively target eosinophilic asthma, Compound 1 is an orally bioavailable small molecule that is effective in treating both human eosinophilic and noneosinophilic (neutrophilic) asthma endotypes.

Various modifications of the invention, in addition to those described herein, will be apparent to those skilled in the art from the foregoing description. Such modifications are also intended to fall within the scope of the appended claims. Each reference, including all patent, patent applications, and publications, cited in the present application is incorporated herein by reference in its entirety.

What is claimed is:

1. A method for treating asthma in a subject, said method comprising administering to the subject a therapeutically effective amount of a JAK1 pathway inhibitor, or a pharmaceutically acceptable salt thereof, wherein the asthma is noneosinophilic asthma.

2. The method of claim 1, wherein the JAK1 pathway inhibitor, or a pharmaceutically acceptable salt thereof, is selective for JAK1 over JAK2, JAK3, and Tyk2.

3. The method of claim 1, wherein the JAK1 pathway inhibitor is 4-[3-(cyanomethyl)-3-(3′,5′-dimethyl-1H,1′H-4,4′-bipyrazol-1-yl)azetidin-1-yl]-2,5-difluoro-N-[(1S)-2,2,2-trifluoro-1-methylethyl]benzamide, or a pharmaceutically acceptable salt thereof.

4. The method of claim 1, wherein the JAK1 pathway inhibitor is 4-[3-(cyanomethyl)-3-(3′,5′-dimethyl-1H,1′H-4,4′-bipyrazol-1-yl)azetidin-1-yl]-2,5-difluoro-N-[(1S)-2,2,2-trifluoro-1-methylethyl]benzamide phosphoric acid salt.

5. The method of claim 1, wherein the JAK1 pathway inhibitor, or pharmaceutically acceptable salt thereof, is administered in a daily dose of about 10 mg to about 80 mg on a free base basis.

6. The method of claim 1, wherein the JAK1 pathway inhibitor, or pharmaceutically acceptable salt thereof, is administered orally.

7. The method of claim 1, wherein the JAK1 pathway inhibitor, or pharmaceutically acceptable salt thereof, is administered orally via tablet.

8. The method of claim 1, wherein the JAK1 pathway inhibitor, or pharmaceutically acceptable salt thereof, is administered in a daily dose of about 15 mg, about 45 mg, or about 75 mg on a free base basis.

9. The method of claim 1, wherein the JAK1 pathway inhibitor, or pharmaceutically acceptable salt thereof, is administered in combination with a further therapeutic agent.

10. The method of claim 9, wherein the further therapeutic agent comprises a Janus kinase inhibitor.

11. The method of claim 10, wherein the Janus kinase inhibitor comprises ruxolitinib, or a pharmaceutically acceptable salt thereof.

12. The method of claim 1, wherein the administering comprises administering the JAK1 pathway inhibitor, or pharmaceutically acceptable salt thereof, together with at least one pharmaceutically acceptable carrier or excipient.

13. The method of claim 1, wherein the subject has about a 10% increase or greater in lung function after administration based on FEV₁.

14. The method of claim 1, wherein the subject has a decrease in a number of inpatient hospitalization events due to asthma, wherein the decrease is about a 30%, about a 60%, or about a 90% reduction in the number of inpatient hospitalization events due to asthma.

15. The method of claim 14, wherein the inpatient hospitalization event comprises admission to an inpatient facility or healthcare facility for ≥24 hours.

16. The method of claim 1, wherein the subject has about a 10% increase or greater in lung function after administration based on FVC.

17. The method of claim 1, wherein the subject has about a 10% improvement or greater in lung function after administration based on FeNO.

18. The method of claim 1, wherein the subject bas about a 10% increase or greater in lung function after administration based on a PEF test result.
